SEQUENCE LISTING (28 sequences)

SEQ ID NO: 1 - DNA, 279 bp, Homo sapiens
aagcaaaggagaagctgagaagatgaaggaaaagtcagggtctggaggggcgggggtcagggagctcctgggagatatggccacatgtagcggctctgaggaatgggttacaggagacctctggggagatgtgaccacagcaatgggtaggagaatgtccagggctatgaaagtcgagtatggggacccccccttaacgaagacagggccatgtagagggccccagggagtgaaagagcctccaggacctccaggtatggaatacaggggacgtttaag

SEQ ID NO: 2 - DNA, 20 bp, Artificial Sequence
  Description of Artificial Sequence  Primer
aagcaaaggagaagctgaga

SEQ ID NO: 3 - DNA, 20 bp, Artificial Sequence
  Description of Artificial Sequence  Primer
cttaaacgtcccctgtattc

SEQ ID NO: 4 - DNA, 26 bp, Artificial Sequence
  Description of Artificial Sequence  Primer
agtcgagtatggggacccccccttaa

SEQ ID NO: 5 - DNA, 22 bp, Artificial Sequence
  Description of Artificial Sequence  Primer
gttaacaatcagcttgccaaat

SEQ ID NO: 6 - DNA, 22 bp, Artificial Sequence
  Description of Artificial Sequence  Primer
cagttctcctccactgccttat

SEQ ID NO: 7 - DNA, 27 bp, Artificial Sequence
  Description of Artificial Sequence  Primer
gcaactcataccagcccatgggtctac

SEQ ID NO: 8 - DNA, 22 bp, Artificial Sequence
  Description of Artificial Sequence  Primer
accttttccatgtggtaactga

SEQ ID NO: 9 - DNA, 24 bp, Artificial Sequence
  Description of Artificial Sequence  Primer
cactaaatcagctttaatcccatt

SEQ ID NO: 10 - DNA, 36 bp, Artificial Sequence
  Description of Artificial Sequence  Primer
ggacactaaatcagctttaatcccattattaagaaa

SEQ ID NO: 11 - DNA, 21 bp, Artificial Sequence
  Description of Artificial Sequence  Primer
ctcccccatgtacttcttcgt

SEQ ID NO: 12 - DNA, 22 bp, Artificial Sequence
  Description of Artificial Sequence  Primer
gcagatcatggagtcaaacaca

SEQ ID NO: 13 - DNA, 26 bp, Artificial Sequence
  Description of Artificial Sequence  Primer
acattgtcaatgtggcgcacaaaggc

SEQ ID NO: 14 - DNA, 22 bp, Artificial Sequence
  Description of Artificial Sequence  Primer
tttgccaacccttaaaatcaat

SEQ ID NO: 15 - DNA, 22 bp, Artificial Sequence
  Description of Artificial Sequence  Primer
gcaggacttcagttccactgtt

SEQ ID NO: 16 - DNA, 34 bp, Artificial Sequence
  Description of Artificial Sequence  Primer
gctatttttagtcagccatgcatttggattttac

SEQ ID NO: 17 - DNA, 28 bp, Artificial Sequence
  Description of Artificial Sequence  Primer
gcagccctgtccaatggaatacaacatt

SEQ ID NO: 18 - DNA, 22 bp, Artificial Sequence
  Description of Artificial Sequence  Primer
tgtttctctcccatcctcactt

SEQ ID NO: 19 - DNA, 22 bp, Artificial Sequence
  Description of Artificial Sequence  Primer
ngtttctctcccatcctcactt

SEQ ID NO: 20 - DNA, 22 bp, Artificial Sequence
  Description of Artificial Sequence  Primer
actttggtggctcgagattcta

SEQ ID NO: 21 - DNA, 26 bp, Artificial Sequence
  Description of Artificial Sequence  Primer
ctctcccatcctcacttcctcaacgc

SEQ ID NO: 22 - DNA, 28 bp, Artificial Sequence
  Description of Artificial Sequence  Primer
cagtcaccgcttctgccagaaccgggtc

SEQ ID NO: 23 - DNA, 33 bp, Artificial Sequence
  Description of Artificial Sequence Synthesized oligo templates
cccctgtatcctgtgtgaaattgttatccgctc

SEQ ID NO: 24 - DNA, 33 bp, Artificial Sequence
  Description of Artificial Sequence Synthesized oligo templates
cccctgtctcctgtgtgaaattgttatccgctc

SEQ ID NO: 25 - DNA, 33 bp, Artificial Sequence
  Description of Artificial Sequence Synthesized oligo templates
cccctgtgtcctgtgtgaaattgttatccgctc

SEQ ID NO: 26 - DNA, 33 bp, Artificial Sequence
  Description of Artificial Sequence Synthesized oligo templates
ddddtgtttcctgtgtgaaattgttatccgctc

SEQ ID NO: 27 - DNA, 24 bp, Artificial Sequence
  Description of Artificial Sequence  Primer
agcggataacaatttcacacagga

SEQ ID NO: 28 - DNA, 22 bp, Artificial Sequence
  Description of Artificial Sequence  Primer
ncagatcatggagtcaaacaca

(19) United States
(12) Patent Application Publication
(10) Pub. No.: US 2003/0027169 A1
(43) Pub. Date: Feb. 6, 2003

(54) One-well assay for high throughput detection of single nucleotide polymorphisms

(76) Inventors: Sheng Zhang, Ithaca, NY (US); Colleen K. Van Pelt, Ithaca, NY (US); Gary A. Schultz, Ithaca, NY (US)

Correspondence Address: Michael L. Goldman, NIXON PEABODY LLP, Clinton Square, P.O. Box 31051, Rochester, NY, 14603, US

(21) Appl. No.: 10/033,300

(22) Filed: Oct. 25, 2001

Related U.S. Application Data
(60) Provisional application No. 60/243,952, filed on Oct. 27, 2000.
(60) Provisional application No. 60/250,434, filed on Dec. 1, 2000.

(51) Int. Cl. C12Q 1/68; C12P 19/34

(52) U.S. Cl. 435/6; 435/91.2

(57) ABSTRACT

The present invention relates to a novel one-well assay which couples a conventional polymerase chain reaction (PCR) amplification step to a single nucleotide primer extension step for the determination of nucleotide sequence variations in the genotyping of single nucleotide polymorphisms and other DNA variations detected by primer extension methods. A PCR amplification step, a phosphatase digestion step (or a molecular weight-selective filter step), and a primer extension step are consecutively performed in the same well plate followed by electrospray mass spectrometry detection of the single nucleotide polymorphism bases. Alternative one-well assays which utilize exonuclease I or λ-exonuclease in addition to the phosphatase digestion step are also disclosed.

This application claims benefit of U.S. Provisional Patent Application Serial No. 60/243,952, filed on Oct. 27, 2000, and No. 60/250,434, filed on Dec. 1, 2000.

### FIELD OF THE INVENTION

The present invention relates to a one-well assay for detection of single nucleotide polymorphisms.

### BACKGROUND OF THE INVENTION

Single-nucleotide polymorphisms (SNPs) are the most frequent type of variation in the human genome with an estimated frequency of one to two polymorphic nucleotides per kilobase (Schafer et al., Nat. Biotechnol., 16: 33-9 (1998); Brookes, Gene, 234: 177-86 (1999)). SNPs can serve as genetic markers for identifying disease genes by linkage studies in families, linkage disequilibrium in isolated populations, association analysis of patients and controls, and loss-of-heterozygosity studies in tumors (Wang et al., Science, 280: 1077-82 (1998)). Although some SNPs in single genes are associated with heritable diseases such as cystic fibrosis, sickle cell anemia, colorectal cancer, and retinitis pigmentosa (Kerem et al., Science, 245: 1073-80 (1989); Fearon et al., Cell, 61: 759-67 (1990); Sung et al., Proc. Natl. Acad. Sci. U.S.A., 88: 6481-5 (1991)), most SNPs are “silent”. They can alter phenotype by either controlling the splicing together of exon from intron-containing genes or changing the way mRNA folds. Recently, there has been increasing knowledge of the genetic basis of SNPs for individual differences in drug response (McCarthy et al., Nat. Biotechnol., 18: 505-8 (2000); Roses, Nature, 405: 857-65 (2000)). Insights into differences between alleles or mutations present in different individuals can also illuminate the interplay of environment with disease susceptibility. For example, in the p53 tumor suppressor gene, over 400 mutations have been found to be associated with tumors and used to determine individuals with increased cancer risk (Kurian et al., J. Pathol., 187: 267-71 (1999)). All these applications involve the analysis of a large number of samples and will eventually require rapid, inexpensive, and highly automated methods for genotyping analysis.

Because of the importance of identifying SNPs, a number of gel-based methods have been described for their detection and genotyping. These methods include single strand conformational polymorphism analysis, heteroduplex analysis, denaturing gradient gel electrophoresis, and chemical or enzyme mismatch modification assays (Schafer and Hawkins, Nat. Biotechnol., 16: 33-9 (1998)). To facilitate large-scale SNP identification, new technologies are being developed to replace the conventional gel-based re-sequencing methods. Perhaps the most widely employed techniques currently used for SNP identification are array hybridization assays, such as allele specific oligonucleotide microarrays in miniaturized assays (Wang et al., Science, 280: 1077-82 (1998)). This approach relies on the capacity to distinguish a perfect match from a single base mismatch by hybridization of target DNA to a related set of four groups of oligonucleotides that are identical except for the base centrally located in the oligonucleotide. Mismatches in the central base of the oligonucleotide sequence have a greater destabilizing effect than mispairing at distal positions during hybridization. Thus, this strategy developed by Affymetrix utilizes a set of four oligonucleotides for each base to re-sequence. For example, a 10-kb gene requires a microarray of 40,000 oligos that can be accomplished by on-chip photolithographic synthesis (Ramsay, Nat. Biotechnol., 16: 40-4 (1998)). The mutation detection is based on the development of a two-color labeling scheme, in which the reference DNA is labeled with phycoerythrin (red) during the PCR amplification, while the target DNA is labeled with fluorescein (green). Both reference and target samples can then be hybridized in parallel to separate chips with identically synthesized arrays or co-hybridized to the same chip. The signal of hybridization of fluorescent products is recorded through confocal microscopy. Comparison of the images for a target sample and reference sample can yield the genotype of the target sample for thousands of SNPs being tested. By processing co-hybridization of the reference and target samples together, experimental variability during the subsequent fragmentation, hybridization, washing, and detection steps can be minimized to make array hybridization more reproducible. The interpretation of the result is based on the ratios between the hybridization signals from the reference and the target DNA with each probe (Hacia et al., Nat. Genet. 14: 441-7 (1996)).

Despite the impressive technology that is emerging for the hybridization to oligonucleotide arrays, potential problems with these approaches exist due to several factors. One limiting factor originates from the inherent properties of the nucleic acid hybridization. The efficiency of hybridization and thermal stability of hybrids formed between the target DNA and a short oligonucleotide probe depend strongly on the nucleotide sequence of the probe and the stringency of the reaction conditions. Furthermore, the degree of destabilization of the hybrid molecule by a mismatched base at one position is dependent on the flanking nucleotide sequence. As a result, it would be difficult to design a single set of hybridization conditions that would provide optimal signal intensities and discrimination of a large number of sequence variants simultaneously. This is particularly true for human genomic DNA which is present either in heterozygous or homozygous form. In addition, the necessity of using DNA chips composed of tens of oligonucleotide probes per analyzed nucleotide position has led to a complex setup of assays and requires mathematical algorithms for interpretation of the data.

Another popular method for high-throughput SNP analysis is called 5′ exonuclease assay in which two fluorogenic probes, double-labeled with a fluorescent reporter dye (FAM or TET) and a quencher dye (TAMRA) are included in a typical PCR amplification (Lee et al., Nucleic Acids Res., 21: 3761-6 (1993); Morin et al., Biotechniques, 27: 538-40, 542, 544passim (1999)). During PCR, the allele-specific probes are cleaved by the 5′ exonuclease activity of Taq DNA polymerase, only if they are perfectly annealed to the segment being amplified. Cleavage of the probes generates an increase in the fluorescence intensity of the reporter dye. As a result, both report fluorescence that can be plotted and segregated to determine the template genotype. The advantage of this approach is to virtually eliminate post-PCR processing. However, the apparent drawbacks of this technique relate to the time and expense of establishing and optimizing conditions for each locus.

Another widely accepted method to identify SNPs is called single nucleotide primer extension (SNuPE), also known as minisequencing (Nikiforov et al., Nucleic Acids Res., 22: 4167-75 (1994); Pastinen et al., Clin. Chem., 42: 1391-17 (1996); Landegren et al., Genome Res., 8: 769-76 (1998)). This technique involves the hybridization of a primer immediately adjacent to the polymorphic locus, extension by a single dideoxynucleotide, and identification of the extended primer. An advantage of this approach, compared to hybridization with oligonucleotide probes, is that all variable nucleotides are identified with optimal discrimination using the same reaction conditions. Consequently, at least one order of magnitude of higher power for discriminating between genotyping is available using this method than with hybridization of allele-specific oligonucleotide probes in the same array format (Pastinen et al., Genome Res., 7: 606-14 (1997)).

Since the first introduction of SNuPE for the identification of genetic disease (Kuppuswamy et al., Proc. Natl. Acad. Sci. U.S.A., 88: 1143-7 (1991)), several new detection methods have been developed including luminous detection (Nyren et al., Anal. Biochem., 208: 171-5 (1993)), colorimetric ELISA (Nikiforov et al., Nucleic Acids Res., 22: 4167-75 (1994)), gel-based fluorescent assays (Pastinen et al., Clin. Chem., 42: 1391-7 (1996)), homogeneous fluorescent detection (Chen et al., Genet. Anal., 14: 157-63 (1999)), flow cytometry-based assays (Cai et al., Genomics, 66: 135-43 (2000)), and high performance liquid chromatography (HPLC) analysis (Hoogendoom et al., Hum. Genet., 104: 89-93 (1999)). Recently, a combination of single nucleotide primer extension and matrix assisted laser desorption ionization-time of flight mass spectrometry (MALDI-TOFMS) detection has been developed (Haff et al., Genome Res., 7: 378-88 (1997); Griffin et al., Trends Biotechnol., 18: 77-84 (2000); Sauer et al., Nucleic Acids Res., 28: E13 (2000)). This approach allows the determination of SNP sequences by measuring the mass difference between the known primer mass and the extended primer mass using MALDI-TOFMS. Discrimination of mass differences of less than 1 part in 1,000 is required to determine which of the four dideoxynucleotide triphosphate bases (ddNTPs), dideoxy-cytidine triphosphate (ddCTP), dideoxy-thymidine triphosphate (ddTTP), dideoxy-adenosine triphosphate (ddATP), and dideoxy-guanosine triphosphate (ddGTP) reacted to extend the primer. A desired capability of this technique includes the analysis of heterozygotes where two different bases are present at the same nucleotide position. The MALDI-TOFMS measurement requires the discrimination of two mass-resolved species that represent the addition of both bases complementary to those at the SNP site. This requires MALDI-TOFMS methods incorporating high mass resolution capabilities and enhanced sensitivity. Compared to the detection of a fluorescence-labeled nucleotide by non-mass spectrometric methods, mass detection is faster, and less laborious without the need for modified or labeled bases. Mass detection offers advantages in accuracy, specificity, and sensitivity. Recently, a chip-based primer extension combined with mass spectrometry detection for genotyping was performed on a 1-μL scale in the wells contained within a microchip without using conventional sample tubes and microtiter plates (Tang et al., Proc. Natl. Acad. Sci. U.S.A., 96: 10016-20 (1999)). This miniaturized method clearly provides another potential for high-throughput and low cost identification of genetic variations.

Another mass spectrometry method that allows for the mass analysis of SNPs is electrospray ionization. Electrospray ionization provides for the atmospheric pressure ionization of a liquid sample by a process that creates highly-charged droplets at atmospheric pressure that, under evaporation, create gas-phase ions representative of the species contained in the solution. The gas-phase ions can be sampled through an ion-sampling orifice of a mass spectrometer for mass selection and detection. Electrospray produces a quantitative response from the MS detector for the analyte molecules present in the liquid.

Most methods utilizing electrospray for the identification of point mutations detect the extended primers. These methods are similar to MALDI-TOFMS in that mass measurements to within 1 part in 1,000 are required to discriminate which base extended the oligonucleotide primer. Also, electrospray ionization of large oligonucleotides is difficult, requiring someone highly skilled in the interpretation of the data.

In order to associate SNPs to any disease (or disease susceptibility) genes, to link SNPs to any individual variability in drug response phenotypes, or to perform genome population studies, the large-scale analysis of hundreds of thousands of SNP samples is required. Currently the most widely used method, mini-sequencing, suffers from the fact that the PCR amplification product, which encompasses the desired SNPs, has to be purified off-line before it can be used as a template for the subsequent primer extension. Not only is PCR product lost in this purification step, but it is also a time-consuming process that is difficult to automate. The significant demands evolving from the modern pharmacogenctics field and growing accumulation of identified SNPs in databases requires much faster, and more accurate, sensitive and effective analytical tools to identify SNPs of individuals for drug development (reviving failed drug, stratifying patient populations and target gene validation).

The present invention is directed to overcoming these deficiencies in the art.

### SUMMARY OF THE INVENTION

The present invention relates to a method of detecting single nucleotide polymorphisms by providing a sample potentially containing a target nucleic acid molecule and subjecting the sample to a polymerase chain reaction process involving use of oligonucleotide amplification primers under conditions effective to amplify any of the target nucleic acid molecule present in the sample to produce an amplification product. The amplification product is then subjected to treatment with a phosphatase under conditions effective to remove 5′ phosphates from free deoxynucleotide triphosphates (dNTPs) in the amplification product, and, then, the phosphatase is inactivated. The amplification product treated with the phosphatase is blended with an oligonucleotide extension primer complementary to a portion of the target nucleic acid molecule, a nucleic acid polymerizing enzyme, and a plurality of types of nucleotide analogs, each type being present in a first amount, to form an extension solution. In the extension solution, the oligonucleotide extension primer is hybridized to the target nucleic acid molecule to form a primed target nucleic acid molecule and the nucleic acid polymerizing enzyme is positioned to add nucleotide analogs to the primed target nucleic acid molecule at an active site. The oligonucleotide extension primer in the extension solution is extended by using the nucleic acid polymerizing enzyme to add a nucleotide analog to the oligonucleotide extension primer at the active site. This forms an extended oligonucleotide extension primer where the nucleotide analog being added is complementary to the nucleotide of the target nucleic acid molecule at the active site. The amounts of each type of the nucleotide analogs in the extension solution after the extending step are then determined where each type is present in a second amount. The first and second amounts of each type of the nucleotide analog are compared. The type of nucleotide analog where the first and second amounts differ is then identified as the nucleotide added to the oligonucleotide extension primer at the active site.

Another aspect of the present invention relates to a method of detecting single nucleotide polymorphisms by providing a sample potentially containing a target nucleic acid molecule and subjecting the sample to a polymerase chain reaction process involving use of oligonucleotide amplification primers under conditions effective to amplify any of the target nucleic acid molecule present in the sample to produce an amplification product. The amplification product is then passed through a molecular weight filter configured to retain amplified target nucleic acid molecule but not the amplification primers. The retained target nucleic acid molecule is blended with an oligonucleotide extension primer complementary to a portion of the target nucleic acid molecule, a nucleic acid polymerizing enzyme, and a plurality of types of nucleotide analogs, each type being present in a first amount, to form an extension solution where the oligonucleotide extension primer is hybridized to the target nucleic acid molecule to form a primed target nucleic acid molecule and the nucleic acid polymerizing enzyme is positioned to add nucleotide analogs to the primed target nucleic acid molecule at an active site. The oligonucleotide extension primer in the extension solution is extended by using the nucleic acid polymerizing enzyme to add a nucleotide analog to the oligonucleotide extension primer at the active site. This forms an extended oligonucleotide extension primer where the nucleotide analog being added is complementary to the nucleotide of the target nucleic acid molecule at the active site. The amounts of each type of the nucleotide analogs in the extension solution after the extending step are then determined where each type is present in a second amount. The first and second amounts of each type of the nucleotide analog are compared. The type of nucleotide analog where the first and second amounts differ is then identified as the nucleotide added to the oligonucleotide extension primer at the active site.

A further aspect of the present invention relates to a method of detecting single nucleotide polymorphisms by providing a sample potentially containing a target nucleic acid molecule and subjecting the sample to a polymerase chain reaction process involving use of oligonucleotide amplification primers under conditions effective to amplify any of the target nucleic acid molecule present in the sample to produce an amplification product. The amplification product is then subjected to treatment with a phosphatase under conditions effective to remove 5′ phosphates from free dNTPs in the amplification product, and, then, the phosphatase is inactivated. The amplification product treated with the phosphatase is blended with an oligonucleotide extension primer complementary to a portion of the target nucleic acid molecule, a nucleic acid polymerizing enzyme, and a plurality of types of nucleotide analogs to form an extension solution where the oligonucleotide extension primer is hybridized to the target nucleic acid molecule to form a primed target nucleic acid molecule and the nucleic acid polymerizing enzyme is positioned to add nucleotide analogs to the primed target nucleic acid molecule at an active site. The oligonucleotide extension primer in the extension solution is extended by using the nucleic acid polymerizing enzyme to add a nucleotide analog to the oligonucleotide extension primer at the active site. This forms an extended oligonucleotide extension primer where the nucleotide analog being added is complementary to the nucleotide of the target nucleic acid molecule at the active site. The nucleotide analog added to the oligonucleotide primer at the active site is then determined by electrospraying the extended oligonucleotide extension primer.

Yet another aspect of the present invention is a method of detecting single nucleotide polymorphisms by providing a sample potentially containing a target nucleic acid molecule, and subjecting the sample to a polymerase chain reaction process involving use of oligonucleotide amplification primers under conditions effective to amplify any of the target nucleic acid molecule present in the sample to produce an amplification product. The amplification product is then passed through a molecular weight filter configured to retain amplified target nucleic acid molecule but not the amplification primers. The retained target nucleic acid molecule is blended with an oligonucleotide extension primer complementary to a portion of the target nucleic acid molecule, a nucleic acid polymerizing enzyme, and a plurality of types of nucleotide analogs to form an extension solution where the oligonucleotide extension primer is hybridized to the target nucleic acid molecule to form a primed target nucleic acid molecule and the nucleic acid polymerizing enzyme is positioned to add nucleotide analogs to the primed target nucleic acid molecule at an active site. The oligonucleotide extension primer in the extension solution is extended by using the nucleic acid polymerizing enzyme to add a nucleotide analog to the oligonucleotide extension primer at the active site. This forms an extended oligonucleotide extension primer where the nucleotide analog being added is complementary to the nucleotide of the target nucleic acid molecule at the active site. The nucleotide analog added to the oligonucleotide extension primer at the active site is then determined by electrospraying the extended oligonucleotide extension primer.

The present invention provides a means to rapidly and accurately identify genetic variations at specific nucleotide positions. This invention has the potential of being employed in any method using primer extension reactions to genotype DNA variations. Further details on methods for detecting single nucleotide polymorphisms can be found in U.S. patent application Ser. No. 09/757,992, which is hereby incorporated in its entirety. There are many different detection methods using primer extension reactions, including but not limited to, MALDI mass spectrometry, electrospray mass spectrometry, fluorescence, spectrophotometry, ion chromatography, liquid chromatography, capillary electrophoresis, nuclear magnetic resonance, colorimeteric ELISA, immuno-radio activity, and radioactivity. In addition, the one-well assay can be incorporated into a variety of other SNP detection methods.

The uniqueness of this one-well analysis resides in the use of PCR amplification primers and single nucleotide extension reaction primers that are designed with significantly different melting temperatures (Tₘ). Different melting temperatures of the designed primers equates to different optimal annealing temperatures applied in both PCR amplification and primer extension reactions. In this one-well assay, the amplification primers are designed with low Tₘ values while the SNP primers (or the extension primers) are designed with high Tₘ values. Consequently, following the PCR amplification step in a one-well reaction, the unreacted amplification primers, which have a low Tₘ value, do not interfere with the subsequent primer extension reaction where a high annealing temperature is used for the high Tₘ value SNP primers. Alternatively, an exonuclease I digestion can be performed following the PCR amplification step. This enzyme digests all single-stranded DNA present in the reaction and thereby eliminates any design constraints for the primers involved in the PCR amplification and the primer extension reactions. Yet another means to overcome any primer constraints is to perform a λ-exonuclease digestion to convert double-stranded PCR product into single-stranded product. In order for λ-exonuclease to be active, one of the amplification primers must be phosphorylated at the 5′ end. Beginning at this 5′ phosphate group, the enzyme progressively digests the one strand involved in the PCR product's DNA duplex. Following the digestion, the only primer remaining has the same directionality as the remaining strand of PCR product, and therefore will not interfere in the primer extension reaction.

The present invention allows for the use of either double or single-stranded DNA to be genotyped. If single-stranded DNA is desired, one of the amplification primers in the PCR amplification is phosphorylated at the 5′-end. Then following the amplification, a λ-exonuclease digestion is performed. During the digestion, λ-exonuclease progressively cleaves the 5′-mononucleotides of only the PCR product strand containing the 5′-phosphate group. The λ-exonuclease enzyme is subsequently inactivated by maintaining the solution temperature at 75° C. for 15 minutes. On the other hand if double-stranded DNA is desired, then the λ-exonuclease digestion can be omitted and non-phosphorylated PCR amplification primers are used.

Alternatively, a plate in 96, 384, 1536, or any other density could be designed which would consist of wells containing a molecular weight filter or metal chelating material. Wells can be positioned as close as 0.1 mm from each other, creating a high-density parallel array reaction well block. The SNP assay is performed directly in this filter plate. Once the reagents for the PCR amplification of genomic DNA are added to the filter plate, the amplification reaction is performed. Following the amplification, a vacuum is applied to the plate, filtering through all the small molecular weight components of the reaction including the unreacted dNTPs, and the amplification primers when the molecular weight filter is permeable to compounds less than 10 kDa. A wash step ensures the complete removal of unreacted dNTPs and amplification primers. At this point the well still contains the PCR products as the molecular weight filter would not permit their permeation. Next, the reagents for the primer extension reaction are added to the wells containing the PCR products. After the primer extension reaction has been performed, a vacuum will once again be applied to the filter plate. The small molecules including the unreacted ddNTPs and magnesium, make their way through the filter, and can be pulled through a bed of metal chelating material. This material is responsible for immobilizing cations such as Mg²⁺ which will interfere in the electrospray ionization process. The effluent from the filter is collected in a clean plate and analyzed. Alternatively, the extended SNP primers that are less than 10 kDa could be detected.

The bed volume of the metal chelating material is large enough to complex the required cations from both the PCR amplification and the primer extension reactions. The filter plate can be designed for use directly in a thermocycler instrument, with vacuum applied to the filter plate either directly in the thermocycler or at an external vacuum manifold. If the molecular weight filter is permeable to compounds less than 10 kDa, then the amplification primer will be washed out of the well following the PCR amplification reaction. This will allow the SNP primer that is used in the subsequent primer extension reaction, to have an unrestricted melting temperature. Conversely, if the molecular weight filter were only permeable to compounds less than 5 kDa, then the amplification primers would not be washed out of the well following the PCR amplification reaction. In this case, it would be necessary for the amplification primers and SNP primers to have significantly different melting temperatures, or to employ either an exonuclease I or a λ-exonuclease digestion. Following the primer extension reaction a vacuum is applied to the plate. The extended primers, remaining in the well, could be detected directly after their reconstitution. If the remaining ddNTPs were to be detected, then the solution would need to pass through a metal chelating material to remove the magnesium.

The present invention provides a means to quantitate a minor or mutant allele frequency in the presence of a second dominant allele present at a higher frequency. This is a particularly useful and powerful technique for disease association and linkage studies where the ability to pool genomic DNA samples and genotype them as if they were single samples will streamline both time and cost. Single-stranded DNA will typically be used in these pooling studies due to an improved primer extension efficiency. Either off-line purified single-stranded DNA or single-stranded DNA from a one-well reaction can be used. A calibration curve is generated, over the percent range of interest of the minor or mutant allele. Then the percent of minor allele can be quantitated within that range of interest in the pooled DNA samples.

The present invention also allows for the amplification of genomic DNA and the primer extension reaction to be performed consecutively within the same reaction container. Furthermore, this robust, reliable assay will have economical advantages over conventional methods. Thus, the present invention not only allows for a sole PCR amplification and primer extension reaction to be carried out in a single well, but it allows for multiple amplifications and multiple primer extension reactions to be performed in a single well. If the SNP is to be detected by the extended primer, this assay can be used in the multiplexing of PCR amplification and primer extension reactions which can be applied to any DNA variation analysis that utilizes a primer extension reaction. In particular, the advantage of this one-well assay is that reaction multiplexing actually occurs in two dimensions. Not only can the individual steps of PCR amplification and primer extension be multiplexed, but the amplification and primer extension reactions themselves are coupled. In addition, conventional purification methods of PCR products routinely generate low yields of purified product. Therefore, by eliminating the need for PCR product purification and the associated product losses, this assay inherently improves the primer extension efficiency, resulting in increased selectivity and sensitivity. Thus, lower amounts of genomic DNA could be used in the assay.

The advantages offered by this method over conventional techniques include its simplicity and its ease of automation. The present invention allows for a point mutation to be determined from genomic DNA in a single-well assay. PCR amplification of genomic DNA and primer extension reactions can be performed in tandem if the amplification primers and SNP primers have significantly different melting temperatures (Tₘ), and if an enzyme with the ability to remove the 5′-phosphates from the unreacted dNTPs is utilized following the PCR amplification reactions. The ability to monitor the activity of the enzyme responsible for removing the 5′-phosphates from the dNTPs is another advantage of this assay. This is possible due to the fact that ddGTP and dATP share the same molecular weight. An intense signal compared to that of the control at the mass corresponding to ddGTP and dATP would indicate the presence of dATP and therefore an incomplete digestion. Alternatively, the PCR amplification and primer extension reactions can be performed in tandem without any primer Tₘ constraints if either a phosphatase and exonuclease I digestion is performed, or if a λ-exonuclease digestion is performed. Exonuclease I is an enzyme with the ability to processively cleave 5′-mononucleotides from single-stranded DNA, destroying the biological activity of any remaining primers in solution. λ-Exonuclease is able to digest 5′-phosphorylated strands of DNA from duplex DNA, thus generating single-stranded DNA. All three enzymes can be inactivated with heat at 75° C. .

The simplicity of the one-well assay results in it being readily amenable to automation, which will render this assay a rapid, reliable, high-throughput method of SNP determination. The assay can be multiplexed which further increases its speed and throughput as well as reducing cost. In addition, this one-well assay eliminates the need for any purification of PCR product which facilitates the primer extension efficiency by avoiding all loss of PCR product, resulting in enhanced selectivity and sensitivity.

The one-well assay disclosed herein is easily amenable to miniaturization. Reaction wells may be designed for reactions of 50 microliters to less than 100 nanoliters. Electrospray mass spectrometry is a concentration sensitive detection method. Thus, the ion response measured by the mass spectrometer is substantially independent of the flow rate at which the sample is analyzed. Detection of ddNTPs by the method disclosed is readily achieved even when the reaction volumes are reduced to less than 100 nanoliter volumes. The cost of this analysis is greatly determined by the amount of each reagent required to perform each of these steps. The ability to perform these reactions in smaller volumes thus greatly reduces the cost, and therefore, the disclosed method provides an improvement over existing methods for SNP detection.

### BRIEF DESCRIPTION OF THE DRAWINGS

FIG. 1 is a schematic diagram of a one-well SNP detection assay in which a PCR amplification, a phosphatase digestion, and a primer extension are consecutively performed in the same reaction well. Following the primer extension reaction the samples are passed through a metal chelating material to remove magnesium, and then the unreacted ddNTPs in each sample are analyzed by electrospray mass spectrometry (ESI/MS/MS).

FIG. 2 is a schematic diagram of a one-well SNP detection assay in which a PCR amplification, a phosphatase and exonuclease I digestion, and a primer extension are consecutively performed in the same reaction well. Following the primer extension reaction, the samples are passed through a metal chelating material to remove magnesium, and then the unreacted ddNTPs in each sample are analyzed by ESI/MS/MS.

FIG. 3 is a schematic diagram of a one-well SNP detection assay in which a PCR amplification, a PCR product purification via a molecular weight filter step, and a primer extension are consecutively performed in the same reaction well. The samples are passed through a metal chelating material to remove magnesium, and then the unreacted ddNTPs in each sample are analyzed by ESI/MS/MS.

FIG. 4 is a schematic diagram of a one-well SNP detection assay in which a PCR amplification, a λ-exonuclease digestion, a phosphatase digestion, and a primer extension are consecutively performed in the same reaction well. Following the primer extension reaction, the samples are passed through a metal chelating material to remove magnesium, and then the unreacted ddNTPs in each sample are analyzed by ESI/MS/MS.

FIG. 5 is a schematic diagram of a one-well SNP detection assay in which a PCR amplification, a λ-exonuclease digestion, a PCR product purification via a molecular weight filter step, and a primer extension are consecutively performed in the same reaction well. The samples are passed through a metal chelating material to remove magnesium, and then the unreacted ddNTPs in each sample are analyzed by ESI/MS/MS.

FIG. 6 is a schematic diagram of a one-well SNP detection assay in which PCR amplification(s), phosphatase digestion, and primer extension(s) are consecutively performed in the same reaction well. The samples are passed through a molecular weight filter to isolate the extended and unextended primers from magnesium and other small molecules, and then the resulting extended primer product(s) are detected by ESI/MS or ESI/MS/MS.

FIG. 7 is a schematic diagram of a one-well SNP detection assay in which a PCR amplification, a phosphatase and exonuclease I digestion, and a primer extension are consecutively performed in the same reaction well. The samples are passed through a molecular weight filter to isolate the extended and unextended primers from magnesium and other small molecules, and then the resulting extended primer product(s) are detected by ESI/MS or ESI/MS/MS.

FIG. 8 is a schematic diagram of a one-well SNP detection assay in which PCR amplification(s), a PCR product purification via a molecular weight filter step, and primer extension(s) are consecutively performed in the same reaction well. The samples are passed through metal chelating material to remove magnesium from the extended and unextended primers. Then the resulting extended primer product(s) are detected by ESI/MS or ESI/MS/MS.

FIG. 9 is a schematic diagram of a one-well SNP detection assay in which PCR amplification(s), λ-exonuclease digestion, phosphatase digestion, and primer extension(s) are consecutively performed in the same reaction well. The samples are passed through a molecular weight filter to isolate the extended and unextended primers from magnesium and other small molecules, and then the resulting extended primer product(s) are detected by ESI/MS or ESI/MS/MS.

FIG. 10 is a schematic diagram of a one-well SNP detection assay in which PCR amplification(s), a λ-exonuclease digestion, a PCR product purification via a molecular weight filter step, and primer extension(s) are consecutively performed in the same reaction well. The samples are passed through a molecular weight filter to isolate the extended and unextended primers from magnesium and other small molecules, and then the resulting extended primer product(s) are detected by ESI/MS or ESI/MS/MS.

FIG. 11A shows a scanning electron micrograph of PVBC/DVB monolith formed by in situ polymerization in the PEEK capillary (500 μm id). The monolith was modified so that its polymer surfaces were grafted with iminodiacetate (IDA) groups.

In FIG. 11B, a schematic shows how the monolithic column is placed between the autosampler and the mass spectrometer to allow for on-line sample preparation.

FIG. 12A shows a cross-sectional view of a two-nozzle electrospray device generating one electrospray plume from each nozzle for one fluid stream.

FIG. 12B shows a cross-sectional view of a two-nozzle electrospray device generating two electrospray plumes from each nozzle for one fluid stream.

FIG. 13 is a schematic diagram for a one-well SNP detection assay that utilizes a filter plate equipped with a molecular weight selective membrane and metal chelating material.

FIGS. 14A-C show devices for detecting single nucleotide polymorphisms according to the present invention.

FIG. 14A shows a reaction well block for performing a reaction, such as polymerase chain reaction and primer extension.

FIG. 14B shows an electrospray system which includes both the reaction well block of FIG. 14A together with an electrospray device.

FIG. 14C depicts an electrospray device with individual wells to which fluid is separately provided by a movable fluid delivery probe.

FIG. 15 shows the DNA base pair sequence of the 279 bp segment of the human TNFα promoter gene used in the one-well assay example. The amplification primers and SNP primer, along with their Tₘ values are shown.

FIG. 16 shows the SRM MS/MS mass spectra of the unreacted ddNTPs following a one-well assay using a human genomic DNA sample with a known heterozygous C/T SNP at −857 of the TNFα gene as a template for PCR amplification. The control sample was concurrently analyzed without adding SNP primer in the primer extension step.

FIG. 17 shows the SRM MS/MS mass spectra of the unreacted ddNTPs following a one-well assay for Region B. The mass spectrum on top is of a control sample where the SNP primer was omitted from the primer extension step. The remaining mass spectra are from human genomic DNA samples with a homozygous G/G SNP (NA 06985A), a homozygous A/A SNP (NA 07349), and a heterozygous G/A SNP (NA 07352).

FIG. 18 shows the SRM MS/MS mass spectra of the unreacted ddNTPs following a one-well assay for Region D, SNP 1. The mass spectrum on top is of a control sample where DNA was omitted. The remaining mass spectra are from human genomic DNA samples with a homozygous C/C SNP (NA 07029), a homozygous T/T SNP (NA07019), and a heterozygous C/T SNP (NA07062).

FIGS. 19A and B are calibration curves for quantitative pooling studies using synthetic templates. In both curves, the minor allele was G while the dominant allele was A.

In FIG. 19A, the calibration curve is over the range of 0% to 30% of oligo G, and the reactions were thermal cycled 60 times.

In FIG. 19B, the calibration curve spans the range of 0% to 100% oligo G, and the reactions were thermal cycled 10 times. The curves were fit to the Michaelis-Menten equation.

FIG. 20 is a calibration curve for a quantitative pooling study using purified single-stranded DNA from Region C of human genomic DNA samples. In this study, T was the minor allele in the presence of the dominant C allele. The curve was fit to the Michaelis-Menten equation and the constants along with their error are provided. In addition to the calibration standards, test samples made by pooling various human genomic DNA samples together were also concurrently analyzed. The theoretical and experimental values of the percent of mutant T allele are provided.

### DETAILED DESCRIPTION OF THE INVENTION

The present invention relates to a method of detecting single nucleotide polymorphisms by providing a sample potentially containing a target nucleic acid molecule and subjecting the sample to a polymerase chain reaction process involving use of oligonucleotide amplification primers under conditions effective to amplify any of the target nucleic acid molecules present in the sample to produce an amplification product. The amplification product is then subjected to treatment with a phosphatase under conditions effective to remove 5′ phosphates from remaining deoxynucleotide triphosphates (dNTPs) in the amplification product, and then the phosphatase is inactivated. The amplification product treated with the phosphates is blended with an oligonucleotide extension primer complementary to a portion of the target nucleic acid molecule, a nucleic acid polymerizing enzyme, and a plurality of types of nucleotide analogs, each type being present in a first amount, to form an extension solution. In the extension solution, the oligonucleotide extension primer is hybridized to the target nucleic acid molecule to form a primed target nucleic acid molecule and the nucleic acid polymerizing enzyme is positioned to add nucleotide analogs to the primed target nucleic acid molecule at an active site. The oligonucleotide extension primer in the extension solution is extended by using the nucleic acid polymerizing enzyme to add a nucleotide analog to the oligonucleotide extension primer at the active site. This forms an extended oligonucleotide extension primer where the nucleotide analog being added is complementary to the nucleotide of the target nucleic acid molecule at the active site. The amounts of each type of the nucleotide analogs in the extension solution after the extending step are then determined where each type is present in a second amount. The first and second amount of each type of the nucleotide analog are compared. The type of nucleotide analog where the first and second amounts differ is then identified as the nucleotide added to the oligonucleotide extension primer at the active site.

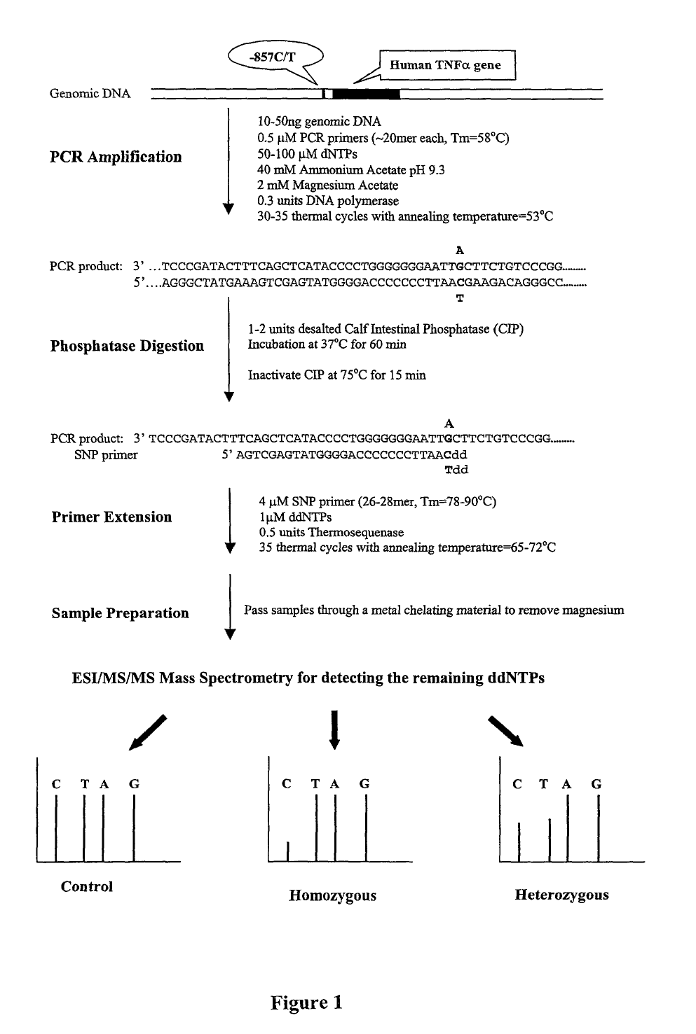

The present invention is a one-well assay for the detection of single nucleotide polymorphisms and other point mutations by ESI/MS. The overall scheme of this method is shown in FIG. 1. The first step in this one-well SNP assay is to amplify by PCR the region of genomic DNA encompassing the particular SNP being investigated. The PCR reaction mixture typically contains 30 to 50 mM of ammonium acetate, 2 mM of magnesium acetate, 0.05-0.2 mM of each of the four deoxynucleotide triphosphates (dNTPs), dATP, dCTP, dGTP, and dTTP, 0.5 μM of both the forward and reverse primers, 1-2 ng/μL of genomic DNA, and 0.3 units of DNA polymerase. The forward and reverse primers used in PCR amplification are typically 20 bases in length with melting temperatures on the order of 58° C. The melting temperature is defined as the temperature at which half of the helical structure is lost. All primers structurally melt or denature over a narrow temperature range that is dictated by their cytosine, guanine, adenine, and thymine content. This narrow melting temperature range, uniquely associated with every primer, makes it possible to design amplification and SNP primers with significantly different annealing temperatures. The reactions are subjected to typical PCR conditions of, for example, 30 cycles with each cycle composed of 95° C. for 1 minute, 50° C. for 30 seconds, and 72° C. for 1 minute.

Following the PCR amplification, an enzyme that has the ability to remove the 5′-phosphates from the unreacted dNTPs remaining in solution is added to the PCR reaction solution. Enzymes such as calf intestinal alkaline phosphatase (CIP) and shrimp alkaline phosphatase (SAP) can be used. The enzyme is incubated in the reaction solution at 37° C. for 30-60 minutes. Finally, the solution is heated to 75° C. for 15 minutes in order to inactivate the enzyme. The purpose of this digestion is to rid the remaining dNTPs of biological activity by removing the 5′ phosphates from the dNTPs so that they will not cause adverse affects in the forthcoming primer extension reaction.

Following the incubation with a phosphatase, the primer extension reaction is performed. The reaction mixtures for primer extension can contain 3-4 μM SNP primer, 1 μM of each of the four dideoxynucleotide triphosphates (ddNTPs), ddATP, ddCTP, ddGTP, and ddTTP, and 5-50 nM synthetic single-stranded DNA or double-stranded PCR product as the target sequence. The SNP primer is present in the reagent composition in a molar excess concentration relative to the nucleotide analog concentrations. A reaction buffer (e.g., 25 mM ammonium acetate, pH 9.3) with 2 mM magnesium acetate and 0.5 units of a Thermosequenase® enzyme (Amersham Pharmacia Biotech Inc., Piscataway, N.J.) may be used for the primer extension reaction. The SNP primer that is to be extended in this step is typically 26 to 28 bases in length with a melting temperature of 78-90° C. The length of the primer is related to its melting temperature, which in turn is related to its annealing temperature requirements in both the PCR amplification and primer extension reactions. In general, the shorter the primer in length, the lower the melting temperature, and the lower the optimal annealing temperature required. Conversely, the longer the primer in length, the higher the melting temperature, and the higher the optimal annealing temperature. If a primer has a low optimal annealing temperature, but a high annealing temperature is used, then the primer will anneal very inefficiently, if at all. Consequently, in this one-well assay, the presence of short, unextended amplification primers in the primer extension reaction will cause no adverse effects during the primer extension step. This is because the primers used in the primer extension reactions are longer and therefore have higher optimal annealing temperatures. Therefore, primers designed with various lengths and consequently various melting temperatures permit the usage of this one-well assay.

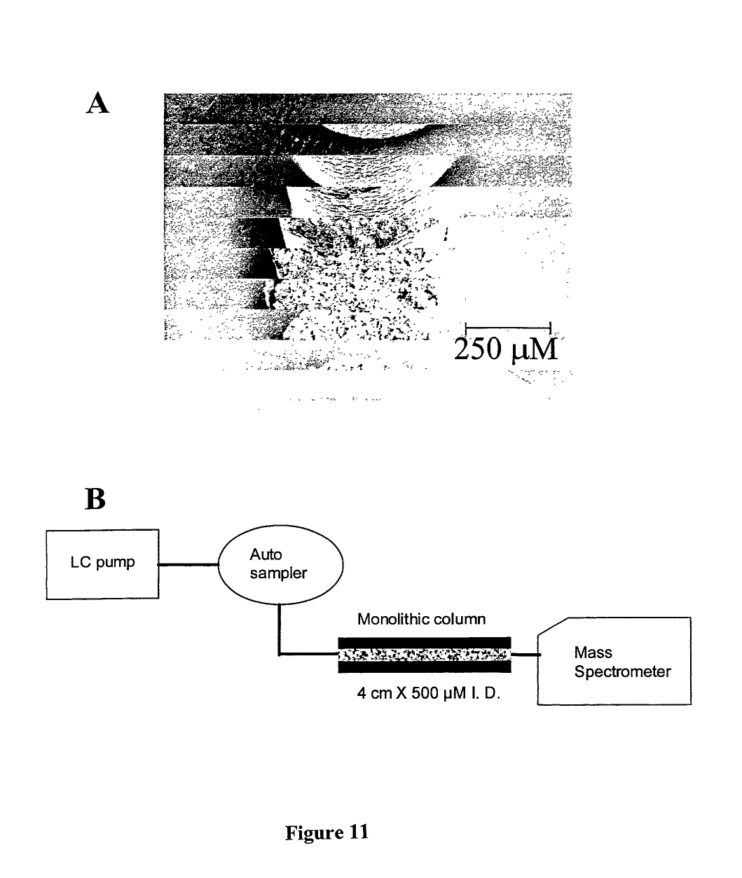

Although a Thermosequenase® enzyme is added to increase the efficiency of the primer extension reaction, this additional enzyme may not ultimately be necessary if a single enzyme is found to carry out both the PCR amplification and the primer extension reactions efficiently. Once formed, the extension solution is subjected to typical primer extension conditions to permit the base added to the 3′ end of the SNP primer to be that which is complementary to the corresponding base in the target nucleotide. Typical primer extension conditions are, for example, 35 cycles composed of 95° C. for 30 seconds, 65-72° C. for 30 sec, and 72° C. for 30 sec. The extension reaction samples are preferably passed through a micro-metal chelating gel column (e.g., immobilized iminodiacetic acid gel from Pierce Chemical Company, Rockford, Ill.) to remove magnesium from the reaction mixture. The resulting samples can then be either directly used for MS analysis or evaporated and reconstituted into distilled water for electrospray mass spectrometry detection of the four ddNTPs. Alternatively, the magnesium can be removed from the reaction solution in an on-line sample preparation technique by passing the sample through a PVBC/DVB monolithic column that has had iminodiacetate (IDA) groups grafted to the surface, as described in U.S. Provisional Patent Application Serial No. 60/269,973, filed on Feb. 20, 2001, which is hereby incorporated by reference in its entirety. FIG. 11A shows a scanning electron micrograph of this column. Upon exiting the column, the sample immediately enters the mass spectrometer, allowing for on-line sample preparation, as shown in FIG. 11B.

The dideoxynucleotide base(s) complementary to the SNP base(s) is substantially consumed (i.e. removed) from the solution during the primer extension reaction. For homozygous SNPs, only one base is substantially consumed whereas for heterozygous SNPs, two bases undergo essentially equal consumption during the thermal cycle extension reaction. In FIG. 1, the base in the target nucleic acid sequence which is susceptible to a single nucleotide polymorphism is either a G or an A. After the primer is extended by one base complementary to the template immediately adjacent to the 3′ end of the primer, thus consuming the nucleotide(s) from the reagent composition, the extension solution is passed through a metal chelating material to remove any magnesium from the solution. The complementary base, which is added to the primer can then be determined by passing the extension solution as well as a control sample through an electrospray device and subjecting the electrospray to mass spectrometry, as set forth in FIG. 1.

This procedure can be used to quantify the concentrations of unreacted ddNTPs remaining in each sample. The advantage of this method is the simplified analysis of the same four analytes used for all possible SNPs. Quantification of free ddNTPs after SNP primer extension reactions may be made by several approaches including but not limited to fluorescence, ion conductivity, liquid chromatography, capillary electrophoresis, mass spectrometry, nuclear magnetic resonance, colorimetric ELISA, immuno-radioactivity (IRA), radioactivity, or any combination thereof. Measurement of the unreacted nucleotide analog concentrations remaining in the reagent solution after primer extension relative to those in a control experiment allows for the immediate determination of the complementary base of the target DNA immediately adjacent to the 3′ end of the oligonucleotide primer.

Preferably, as shown in FIG. 1, using mass spectrometry, the relative ion intensity for each of the nucleotide analogs is determined for each sample. By comparing the relative ion intensity of the extension solution and the control sample, the complementary base can be determined. In particular, that base is the base present in the extension solution in an amount that is less than that present in the control sample. As shown in FIG. 1, the control sample has equal relative intensities for each of the nucleotide analogs. When the sample is homozygous for the target nucleic acid sequence with a G at the polymorphism site, the relative intensity for the complementary base, C, is lower than for the other nucleotide analogs, as shown in FIG. 1. On the other hand, when the sample is heterozygous for the target nucleic acid sequence with a G and A at the polymorphism site, the relative intensity for the complementary bases, C and T, respectively, is lower than for the other nucleotide analogs, as shown in FIG. 1.

An alternative means of preventing the remaining amplification primers from annealing to the target DNA is to add exonuclease I together with phosphatase to the reaction well after the amplification step. Exonuclease I progressively cleaves 5′-monophosphates from single-stranded DNA without affecting double-stranded DNA. Thus, exonuclease I digests the left-over amplification primers. After treatment, both the phosphatase and exonuclease I enzymes are inactivated by heating the reaction to 75° C. and maintaining that temperature for 15 minutes, before the subsequent extension step.

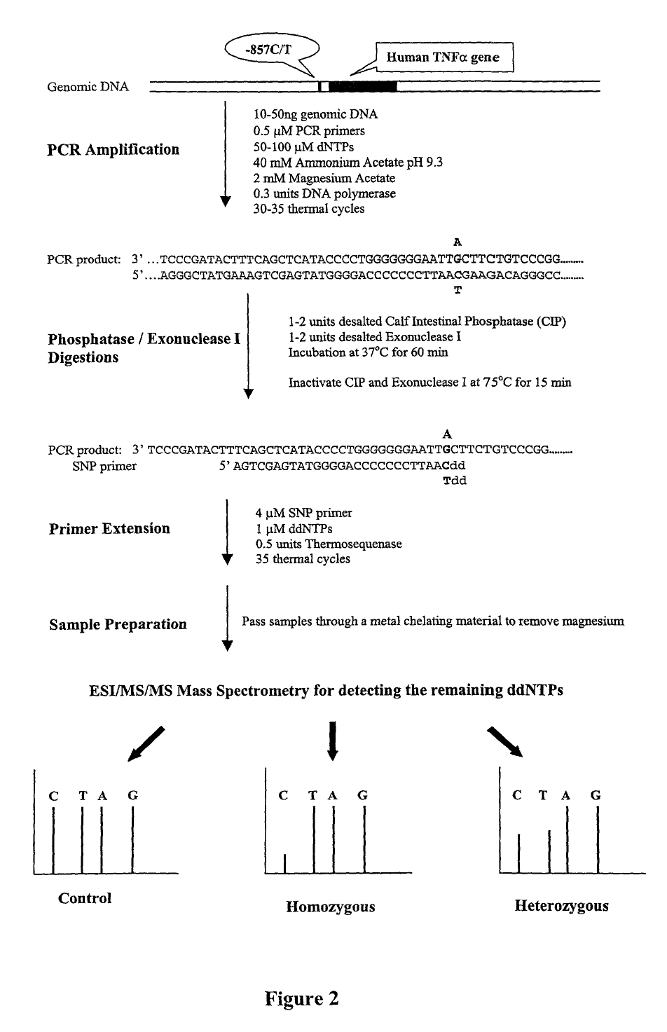

FIG. 2 shows the steps involved in this embodiment of the present invention where exonuclease I is introduced. The first step in this one-well SNP assay is to amplify the region of genomic DNA encompassing the particular SNP being investigated. This PCR reaction contains 30 to 50 mM of ammonium acetate, 2 mM of magnesium acetate, 0.05-0.10 mM of each of the four deoxynucleotide triphosphates (dNTPs), dATP, dCTP, dGTP, and dTTP, 0.5 μM of both the forward and reverse primers, 1-2 ng/μL of genomic DNA, and 0.3 units of DNA polymerase. The total volume of this amplification reaction has been done in both 50 and 10 μL, and it can certainly be further reduced. The forward and reverse primers used in this PCR amplification are 20-22 bases in length with melting temperatures on the order of 58° C. The reactions are subjected to typical PCR conditions of 35 cycles with each cycle composed of 95° C. for 1 minute, 52° C. for 45 seconds, and 72° C. for 45 seconds.

Following the PCR amplification, the phosphatase (e.g., CIP) and the exonuclease I are added to remove the 5′-phosphates from the unreacted dNTPs and amplification primers remaining in solution. The enzymes are incubated in the reaction solution at 37° C. for 30-60 minutes. Finally, the solution is heated to 75° C. for 15 minutes in order to inactivate the enzyme. The purpose of this digestion is to rid the remaining dNTPs of biological activity as well as to digest the remaining amplification primers so that neither component will cause adverse affects in the forthcoming primer extension reaction.

Following the incubation, the primer extension reaction is performed. To the reaction mixture is added 1 μM of each of the four dideoxynucleotide triphosphates (ddNTPs), ddATP, ddCTP, ddGTP, and ddTTP, as well as 4 μM of SNP primer, and 0.5 units of Thermosequenase® enzyme. The primer that is to be extended in this step is 20-26 bases in length with a melting temperature of 60-90° C. Typical primer extension conditions would consist of 30-35 cycles composed of 95° C. for 30 seconds, 60-72° C. (dependent on the SNP primer Tₘ) for 30 seconds, and 72° C. for 30 seconds. This thermal cycle for primer extension reaction could be cut down to as low as 5-10 cycles. The reactions could then be subjected to sample clean-up steps if required by the detection method, or immediately analyzed. The PCR amplification and primer extension steps are consecutively performed followed by ESI/MS/MS detection of the SNP bases. This one-well assay can be used with any SNP detection method that uses primer extension reactions.

The advantage of the exonuclease I method in a one-well assay is that it can preclude the limitation of the design requirement of amplification and primer extension oligonucleotide primers with different Tₘ values. This can be useful since occasionally the target DNA region may not permit design of short amplification primers. The disadvantage is that it introduces another enzyme cost although the cost will be diminished when the miniaturization analysis system is applied.

Another aspect of the present invention relates to a method of detecting single nucleotide polymorphisms by providing a sample potentially containing a target nucleic acid molecule and subjecting the sample to a polymerase chain reaction process involving use of oligonucleotide amplification primers under conditions effective to amplify any of the target nucleic acid molecules present in the sample to produce an amplification product. The amplification product is then passed through a molecular weight filter configured to retain amplified target nucleic acid molecules, but not the amplification primers. The retained target nucleic acid molecules are blended with an oligonucleotide extension primer complementary to a portion of the target nucleic acid molecule, a nucleic acid polymerizing enzyme, and a plurality of types of nucleotide analogs, each type being present in a first amount, to form an extension solution where the oligonucleotide extension primer is hybridized to the target nucleic acid molecule to form a primed target nucleic acid molecule and the nucleic acid polymerizing enzyme is positioned to add nucleotide analogs to the primed target nucleic acid molecule at an active site. The oligonucleotide extension primer in the extension solution is extended by using the nucleic acid polymerizing enzyme to add a nucleotide analog to the oligonucleotide extension primer at the active site. This forms an extended oligonucleotide extension primer where the nucleotide analog being added is complementary to the nucleotide of the target nucleic acid molecule at the active site. The amounts of each type of the nucleotide analogs in the extension solution after the extending step are then determined where each type is present in a second amount. The first and second amount of each type of the nucleotide analog are compared. The type of nucleotide analog where the first and second amounts differ is then identified as the nucleotide added to the oligonucleotide extension primer at the active site.

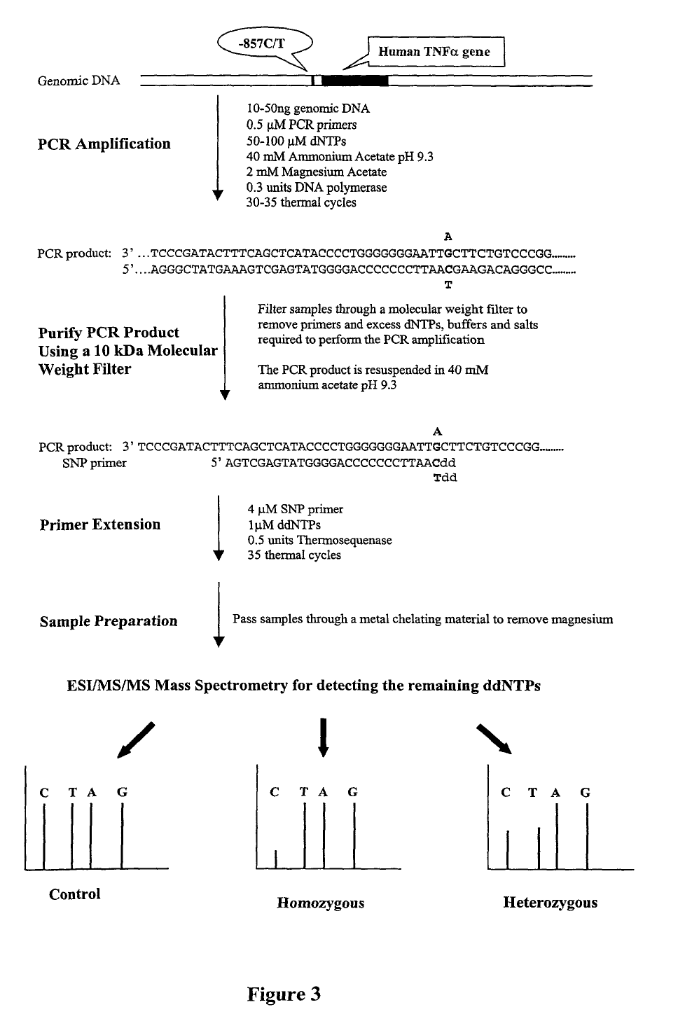

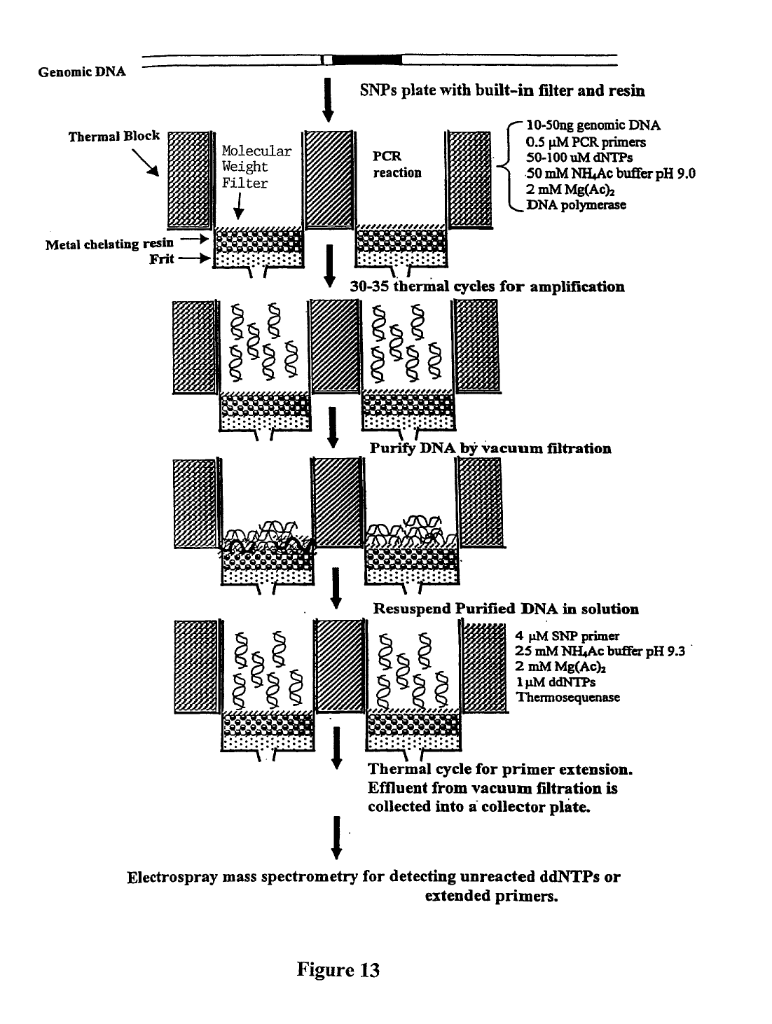

FIG. 3 shows the overall scheme of this alternative one-well SNP assay. Thus, instead of treating the PCR reaction solution with a phosphatase after the PCR amplification step, the reaction solution is filtered through a molecular weight filter designed to retain the amplified DNA but allow the amplification primers and unreacted dNTPs to pass through. The amplified DNA is washed sufficiently with buffer solution to adequately remove the amplification primers and dNTPs. Following the washing step, the amplified DNA is resuspended in a buffer. An SNP primer, ddNTPs, and a Thermosequenase® enzyme or other appropriate enzyme are combined and thermal cycled to perform the primer extension reaction. The extension reaction samples are preferably passed through a micro-metal chelating gel column (e.g., immobilized iminodiacetic acid gel from Pierce Chemical Company, Rockford, Ill.) to remove magnesium from the reaction mixture. The resulting samples can then be either directly used for MS analysis or evaporated and reconstituted into distilled water for electrospray mass spectrometry detection of the four ddNTPs. Alternatively, the magnesium can be removed from the reaction solution in an on-line sample preparation technique by passing the sample though a PVBC/DVB monolithic column that has had iminodiacetate (IDA) groups grafted to the surface, as described in U.S. Provisional Patent Application Serial No. 60/269,973, filed on Feb. 20, 2001, which is hereby incorporated by reference in its entirety. See also FIG. 13.

In another embodiment of the present invention, a sample potentially containing a target nucleic acid molecule is provided and the sample is subjected to a polymerase chain reaction process involving use of oligonucleotide amplification primers, one of which is phosphorylated at the 5′-end, under conditions effective to amplify any of the target nucleic acid molecules present in the sample to produce an amplification product. The amplification product is then subjected to treatment with λ-exonuclease under conditions effective to digest the one strand of PCR product containing the 5′-phosphate group. Following the λ-exonuclease digestion, the amplification product is subjected to treatment with a phosphatase under conditions effective to remove 5′-phosphates from remaining dNTPs in the amplification solution, and, then the λ-exonuclease and phosphatase are inactivated. The amplification solution then is blended with an oligonucleotide extension primer complementary to a portion of the target nucleic acid molecule, a nucleic acid polymerizing enzyme, and a plurality of types of nucleotide analogs, each type being present in a first amount, to form an extension solution. In the extension solution, the oligonucleotide extension primer is hybridized to the target nucleic acid molecule to form a primer target nucleic acid molecule and the nucleic acid polymerizing enzyme is positioned to add nucleotide analogs to the primed target nucleic acid molecule at an active site. The oligonucleotide extension primer in the extension solution is extended by using the nucleic acid polymerizing enzyme to add a nucleotide analog to the oligonucleotide extension primer at the active site. This forms an extended oligonucleotide extension primer where the nucleotide analog being added is complementary to the nucleotide of the target nucleic acid molecule at the active site. The amounts of each type of the nucleotide analogs in the extension solution after the extension step are then determined where each type is present in a second amount. The first and second amounts of each type of the nucleotide analog are compared. The type of nucleotide analog where the first and second amounts differ is then identified as the nucleotide added to the oligonucleotide extension primer at the active site.

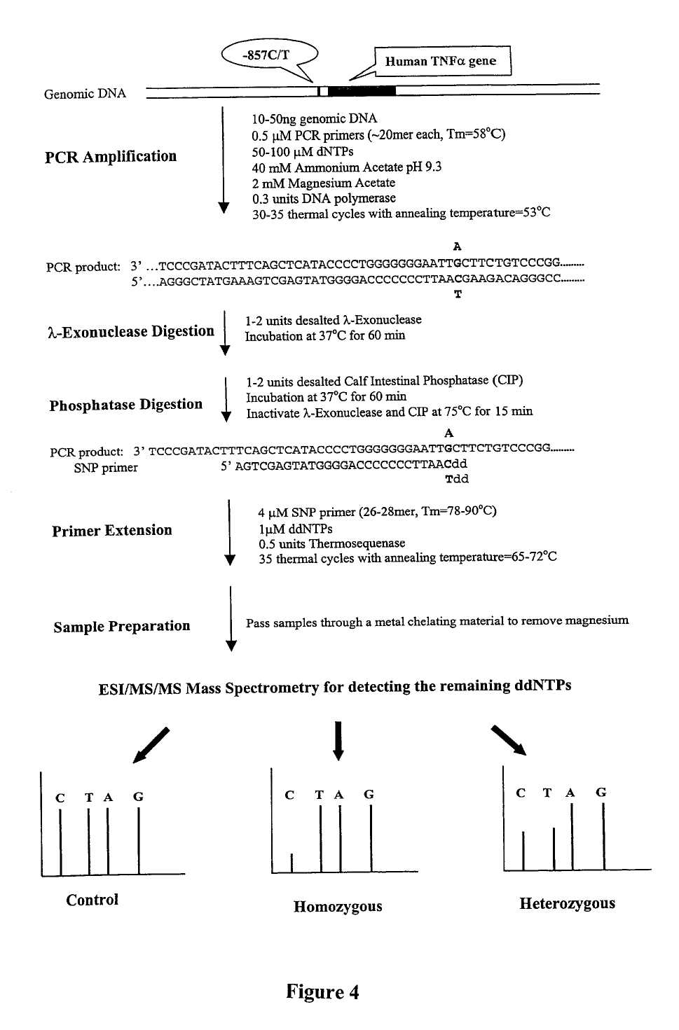

FIG. 4 shows the overall scheme of this embodiment of the present invention. Immediately following the PCR amplification, a λ-exonuclease digestion is performed using 1 to 2 units of desalted enzyme. The reaction is incubated for 1 hour at 37° C. During this incubation period, the enzyme converts the double-stranded PCR product into single-strand DNA, by progressively cleaving mononucleotides starting at the 5′-phosphorylation site. Next, a phosphatase digestion is performed by adding 1 to 2 units of desalted enzyme and allowing the reaction to incubate at 37° C. for 1 hour. During this period, the 5′ phosphates are removed from the unreacted dNTPs remaining in solution. Next, both λ-exonuclease and phosphatase are inactivated by heating the reaction at 75° C. for 15 min. Following the digestions, SNP primer, ddNTPs, and Thermosequenase® enzyme or other appropriate enzymes are added and thermal cycled to perform the primer extension reaction. The reaction solutions can be passed through a metal chelating material to effectively remove the magnesium from the solution, and the unreacted ddNTPs are collected for analysis by electrospray mass spectrometry.

In another embodiment of the present invention, a sample containing a target nucleic acid molecule is provided and the sample is subjected to a polymerase chain reaction process involving use of oligonucleotide amplification primers, one of which is phosphorylated at the 5′-end, under conditions effective to amplify any of the target nucleic acid molecules present in the sample to produce an amplification product. The amplification product is then subjected to treatment with λ-exonuclease under conditions effective to digest the one strand of PCR product containing the 5′-phosphate group. The λ-exonuclease is then inactivated. Then, the amplification solution is passed though a molecular weight filter configured to retain amplified target nucleic acid molecule, but not the amplification primer. The retained target nucleic acid molecule is blended with an oligonucleotide extension primer complementary to a portion of the target nucleic acid molecule, a nucleic acid polymerizing enzyme, and a plurality of types of nucleotide analogs, each type being present in a first amount, to form an extension solution where the oligonucleotide extension primer is hybridized to the target nucleic acid molecule to form a primed target nucleic acid molecule and the nucleic acid polymerizing enzyme is positioned to add nucleotide analogs to the primed target nucleic acid molecule at an active site. The oligonucleotide extension primer in the extension solution is extended by using the nucleic acid polymerizing enzyme to add a nucleotide analog to the oligonucleotide extension primer at the active site. This forms an extended oligonucleotide extension primer where the nucleotide analog being added is complementary to the nucleotide of the target nucleic acid molecule at the active site. The reaction solution is passed through a metal chelating material to remove magnesium. The amounts of each type of the nucleotide analogs in the extension solution after the extending step are then determined where each type is present in a second amount. The first and second amounts of each type of the nucleotide analog are compared. The type of nucleotide analog where the first and second amounts differ is then identified as the nucleotide added to the oligonucleotide extension primer at the active site.

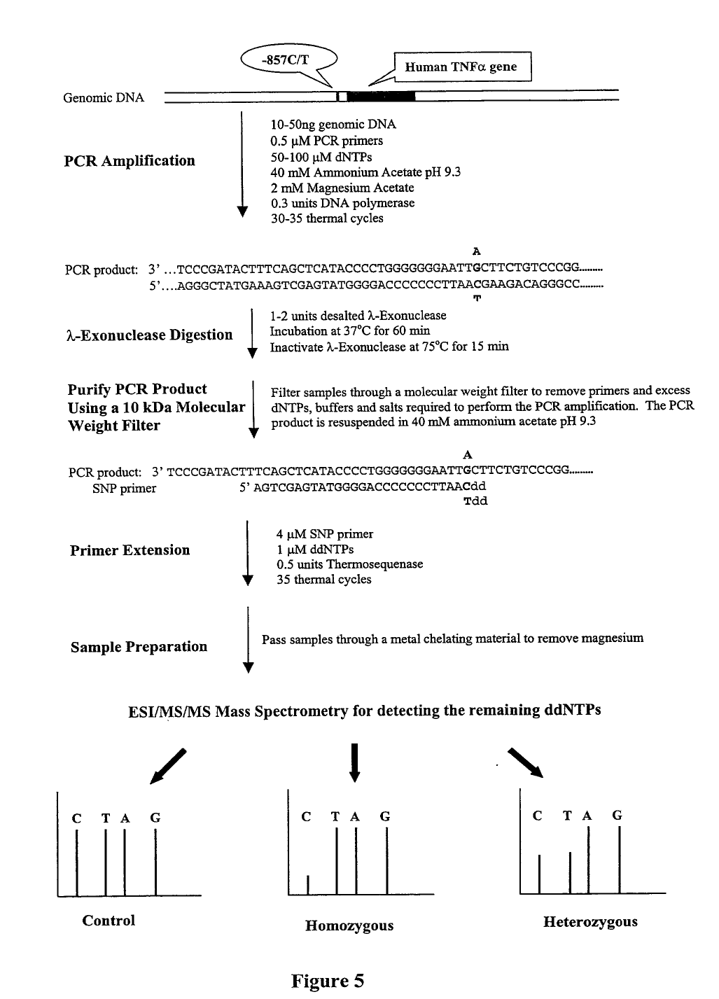

FIG. 5 shows the overall scheme of this embodiment of the present invention. Immediately following the PCR amplification, a λ-exonuclease digestion is performed using 1 to 2 units of desalted enzyme. The reaction is incubated for 1 hour at 37° C. During this incubation period, the enzyme converts the double-stranded PCR product into single-strand DNA, by progressively cleaving mononucleotides starting at the 5′-phosphorylation site. The enzyme is then inactivated by heating the reaction at 75° C. for 15 min. Then, instead of treating the reaction solution with a phosphatase, it is filtered through a molecular weight filter designed to retain the amplified DNA but allow the amplification primers and unreacted dNTPs to pass through. The amplified DNA is washed sufficiently with buffer solution to adequately remove the amplification primers and dNTPs. Following the washing step, the amplified DNA is resuspended in a buffer. Then SNP primer, ddNTPs, and Thermosequenase® enzyme or other appropriate enzymes are combined and thermal cycled to perform the primer extension reaction. The reaction solutions can be passed through a metal chelating material to effectively remove the magnesium from the solution, and the unreacted ddNTPs are collected for analysis by electrospray mass spectrometry.

A further aspect of the present invention relates to a method of detecting single nucleotide polymorphisms by providing a sample potentially containing a target nucleic acid molecule and subjecting the sample to a polymerase chain reaction process involving use of oligonucleotide amplification primers under conditions effective to amplify any of the target nucleic acid molecules present in the sample to produce an amplification product. The amplification product is then subjected to treatment with a phosphatase under conditions effective to remove 5′-phosphates from free dNTPs in the amplification solution, and then the phosphatase is inactivated. The amplification product treated with the phosphatase is blended with an oligonucleotide extension primer complementary to a portion of the target nucleic acid molecule, a nucleic acid polymerizing enzyme, and a plurality of types of nucleotide analogs to form an extension solution where the oligonucleotide extension primer is hybridized to the target nucleic acid molecule to form a primed target nucleic acid molecule and the nucleic acid polymerizing enzyme is positioned to add nucleotide analogs to the primed target nucleic acid molecule at an active site. The oligonucleotide extension primer in the extension solution is extended by using the nucleic acid polymerizing enzyme to add a nucleotide analog to the oligonucleotide extension primer at the active site. This forms an extended oligonucleotide extension primer where the nucleotide analog being added is complementary to the nucleotide of the target nucleic acid molecule at the active site. The nucleotide analog added to the oligonucleotide primer at the active site is then determined by electrospraying the extended oligonucleotide extension primer.

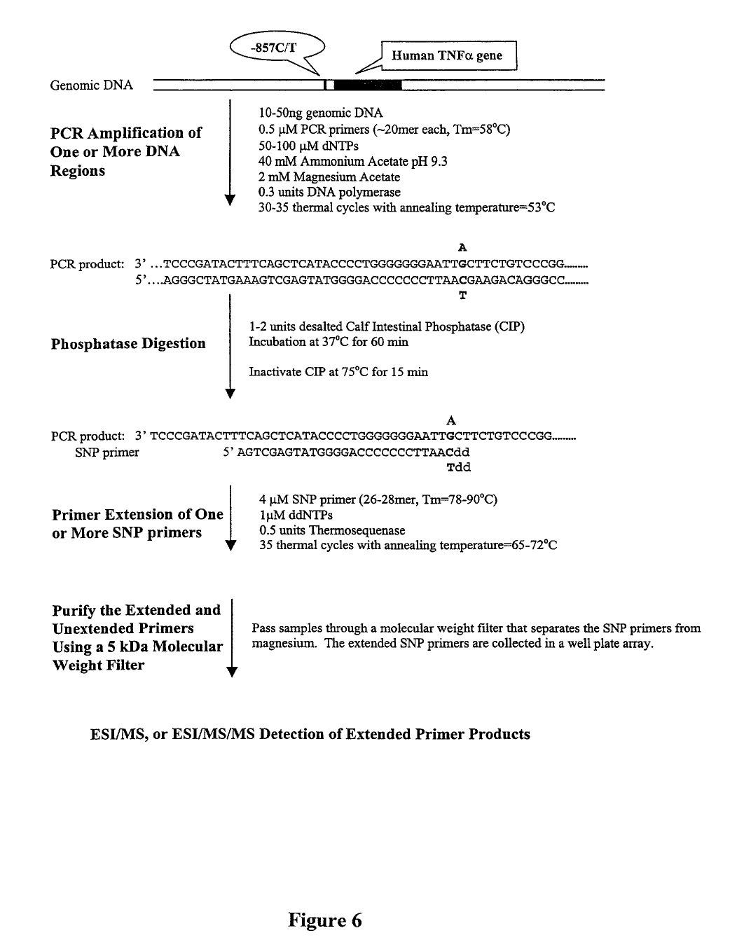

FIG. 6 shows the overall scheme of this alternative one-well SNP assay. After the PCR amplification step, the reaction solution is incubated with phosphatase to remove the 5′-phosphates from the unreacted dNTPs in the solution. Following the incubation, the primer extension reaction is performed. The extension solution can then be passed though a molecular weight filter to separate the amplified target nucleic acid molecule and the extended oligonucleotide extension primer from the low molecular weight reaction components. The nucleotide analog added to the oligonucleotide primer at the active site is then determined by electrospraying the extended oligonucleotide extension primer.

In another embodiment of the present invention, a sample containing a target nucleic acid molecule is provided and the sample is subjected to a polymerase chain reaction process involving use of oligonucleotide amplification primers under conditions effective to amplify any of the target nucleic acid molecules present in the sample to produce an amplification product. The amplification solution is then subjected to treatment with a phosphatase under conditions effective to remove 5′-phosphates from remaining dNTPs in the amplification solution. The amplification solution is simultaneously treated with exonuclease I under conditions effective to digest remaining single-stranded primers. Then, the phosphatase and exonuclease I are inactivated. The amplification solution treated with the phosphatase and exonuclease I is blended with an oligonucleotide extension primer complementary to a portion of the target nucleic acid molecule, a nucleic acid polymerizing enzyme, and a plurality of types of nucleotide analogs, each type being present in a first amount, to form an extension solution. In the extension solution, the olignucleotide extension primer is hybridized to the target nucleic acid molecule to form a primed target nucleic acid molecule and the nucleic acid polymerizing enzyme is positioned to add nucleotide analogs to the primed target nucleic acid molecule at an active site. The oligonucleotide extension primer in the extension solution is extended by using the nucleic acid polymerizing enzyme to add a nucleotide analog to the oligonucleotide extension primer at the active site. This forms an extended oligonucleotide extension primer where the nucleotide analog being added is complementary to the nucleotide of the target nucleic acid molecule at the active site. The extension solution can then be passed though a molecular weight filter to separate the amplified target nucleic acid molecule and the extended oligonucleotide extension primer from the low molecular weight reaction components. The nucleotide analog added to the oligonucleotide primer at the active site is then determined by electrospraying the extended oligonucleotide extension primer.

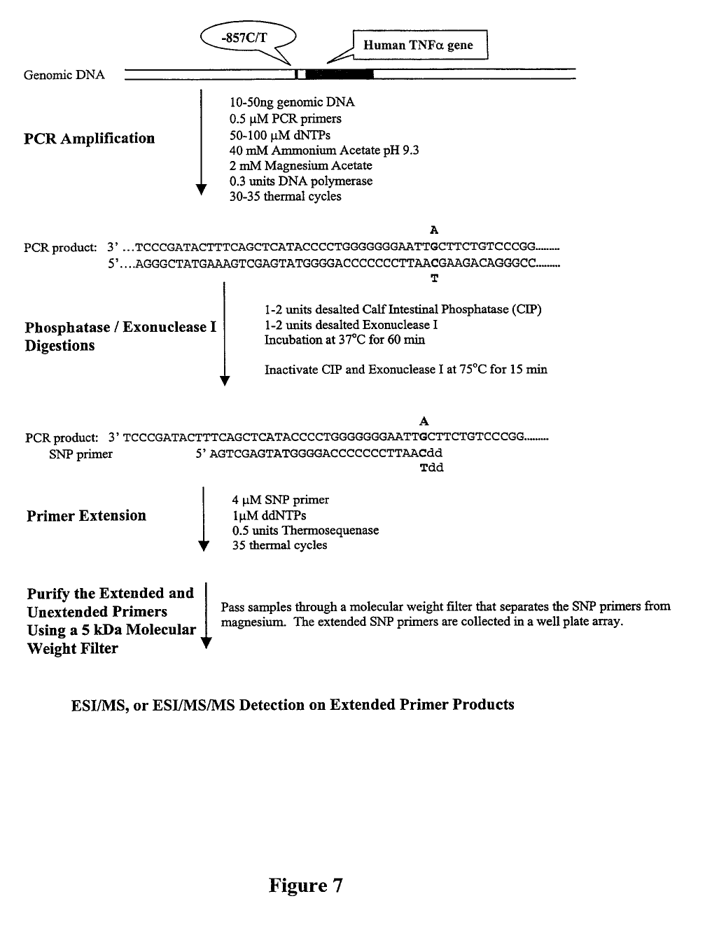

FIG. 7 shows the overall scheme of this alternative one-well SNP assay. Immediately following the PCR amplification, a phosphatase and exonuclease I digestion is performed using 1 to 2 units of each desalted enzyme. The reaction is incubated for 1 hour at 37° C. During this period the 5′-phosphates are removed from the unreacted dNTPs remaining in solution by the phosphatase, and the single-stranded primers were degraded by the exonuclease I. Next, both phosphatase and exonuclease I are inactivated by heating the reaction at 75° C. for 15 min. Following the digestions, SNP primer, ddNTPs, and Thermosequenase® enzyme or other appropriate enzymes are combined and thermal cycled to perform the primer extension reaction. The extension solution can then be passed though a molecular weight filter to separate the amplified target nucleic acid molecule and the extended oligonucleotide extension primer from the low molecular weight reaction components. The nucleotide analog added to the oligonucleotide primer at the active site is then determined by electrospraying the extended oligonucleotide extension primer.

Another aspect of the present invention is a method of detecting single nucleotide polymorphisms by providing a sample potentially containing a target nucleic acid molecule, and subjecting the sample to a polymerase chain reaction process involving use of oligonucleotide amplification primers under conditions effective to amplify any of the target nucleic acid molecules present in the sample to produce an amplification product. The amplification product is then passed through a molecular weight filter configured to retain amplified target nucleic acid molecules, but not the amplification primers. The retained target nucleic acid molecule is blended with an oligonucleotide extension primer complementary to a portion of the target nucleic acid molecule, a nucleic acid polymerizing enzyme, and a plurality of types of nucleotide analogs to form an extension solution where the oligonucleotide extension primer is hybridized to the target nucleic acid molecule to form a primed target nucleic acid molecule and the nucleic acid polymerizing enzyme is positioned to add nucleotide analogs to the primed target nucleic acid molecule at an active site. The oligonucleotide extension primer in the extension solution is extended by using the nucleic acid polymerizing enzyme to add a nucleotide analog to the oligonucleotide extension primer at the active site. This forms an extended oligonucleotide extension primer where the nucleotide analog being added is complementary to the nucleotide of the target nucleic acid molecule at the active site. The nucleotide analog added to the oligonucleotide extension primer at the active site is then determined by electrospraying the extended oligonucleotide extension primer.

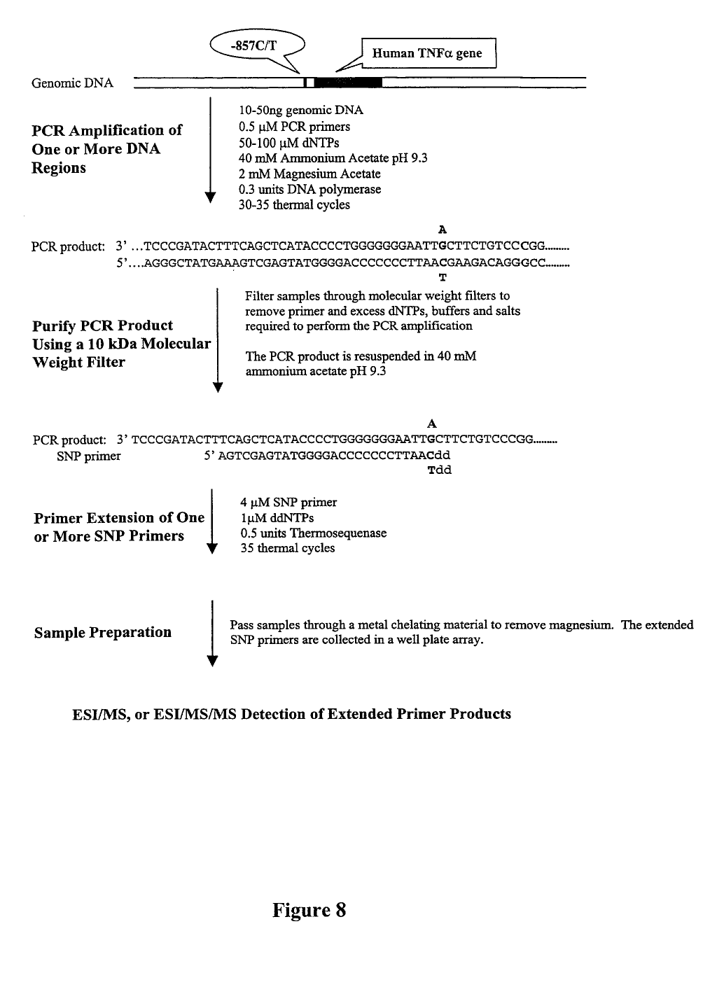

FIG. 8 shows the overall scheme of this alternative one-well SNP assay. After the PCR amplification step, the reaction solution is filtered through a molecular weight filter designed to retain the amplified DNA but allow the amplification primers and unreacted dNTPs to pass through. The amplified DNA is washed sufficiently with buffer solution to adequately remove the amplification primers and dNTPs. Following the washing step, the amplified DNA is resuspended in a buffer. SNP primer, ddNTPs, and a Thermosequenase® nucleic acid polymerizing enzyme or another appropriate enzyme are combined and thermal cycled to perform the primer extension reaction. After the primer extension step, the reaction solutions can be treated with metal chelating material. The extended SNP primers are then analyzed by electrospray mass spectrometry.

In another embodiment of the present invention, a sample potentially containing a target nucleic acid molecule is provided and the sample is subjected to a polymerase chain reaction process involving use of oligonucleotide amplification primers, one of which is phosphorylated at the 5′-end, under conditions effective to amplify any of the target nucleic acid molecules present in the sample to produce an amplification product. The amplification product is then subjected to treatment with λ-exonuclease under conditions effective to digest the one strand of PCR product containing the 5′-phosphate group. Following the λ-exonuclease digestion, the amplification product is subjected to treatment with a phosphatase under conditions effective to remove 5′-phosphates from free dNTPs in the amplification solution, and, then, both the λ-exonuclease and the phosphatase are inactivated. The amplification product treated with the phosphatase is blended with an oligonucleotide extension primer complementary to a portion of the target nucleic acid molecule, a nucleic acid polymerizing enzyme, and a plurality of types of nucleotide analogs to form an extension solution where the oligonucleotide extension primer is hybridized to the target nucleic acid molecule to form a primed target nucleic acid molecule and the nucleic acid polymerizing enzyme is positioned to add nucleotide analogs to the primed target nucleic acid molecule at an active site. The oligonucleotide extension primer in the extension solution is extended by using the nucleic acid polymerizing enzyme to add a nucleotide analog to the oligonucleotide extension primer at the active site. This forms an extended oligonucleotide extension primer where the nucleotide analog being added is complementary to the nucleotide of the target nucleic acid molecule at the active site. The extension solution can then be passed through a molecular weight filter to separate the amplified target nucleic acid molecule and the extended oligonucleotide extension primer from the low molecular weight reaction components. The nucleotide analog added to the oligonucleotide primer at the active site is then determined by electrospraying the extended oligonucleotide extension primer.

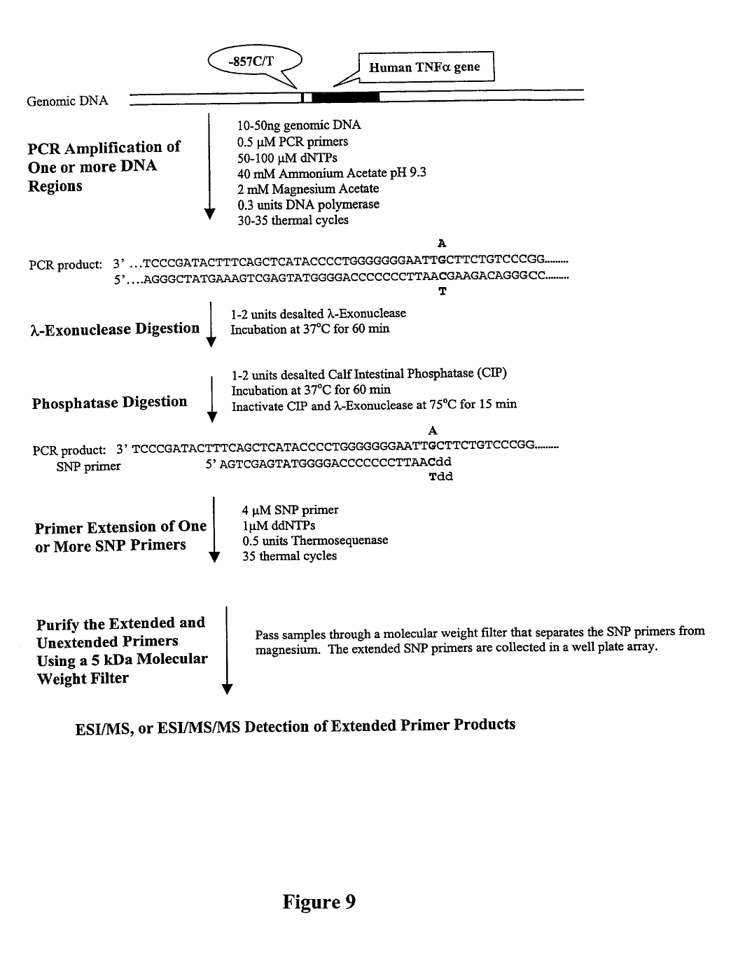

FIG. 9 shows the overall scheme of this embodiment of the present invention. Immediately following the PCR amplification, a λ-exonuclease digestion is performed using 1 to 2 units of desalted enzyme. The reaction is incubated for 1 hour at 37° C. During this incubation period, the enzyme converts the double-stranded PCR product into single-strand DNA, by progressively cleaving mononucleotides starting at the 5′-phosphorylation site. Next, a phosphatase digestion is performed by adding 1 to 2 units of desalted enzyme and allowing the reaction to incubate at 37° C. for 1 hour. During this period, the 5′-phosphates are removed from the unreacted dNTPs remaining in solution. Next, both λ-exonuclease and phosphatase are inactivated by heating the reaction at 75° C. for 15 min. Following the digestions, SNP primer, ddNTPs, and Thermosequenase® enzyme or other appropriate enzymes are combined and thermal cycled to perform the primer extension reaction. The extension solution can then be passed though a molecular weight filter to separate the amplified target nucleic acid molecule and the extended oligonucleotide extension primer from the low molecular weight reaction components. The nucleotide analog added to the oligonucleotide primer at the active site is then determined by electrospraying the extended oligonucleotide extension primer.

In another embodiment of the present invention, a sample potentially containing a target nucleic acid molecule is provided and the sample is subjected to a polymerase chain reaction process involving use of oligonucleotide amplification primers, one of which is phosphorylated at the 5′-end, under conditions effective to amplify any of the target nucleic acid molecules present in the sample to produce an amplification product. The amplification product is then subjected to treatment with λ-exonuclease under conditions effective to digest the one strand of PCR product containing the 5′-phosphate group. The λ-exonuclease is then inactivated. Then, the amplification product is passed through a molecular weight filter configured to retain amplified target nucleic acid molecules. The retained target nucleic acid molecule is blended with an oligonucleotide extension primer complementary to a portion of the target nucleic acid molecule, a nucleic acid polymerizing enzyme, and a plurality of types of nucleotide analogs to form an extension solution where the oligonucleotide extension primer is hybridized to the target nucleic acid molecule to form a primed target nucleic acid molecule and the nucleic acid polymerizing enzyme is positioned to add nucleotide analogs to the primed target nucleic acid molecule at an active site. The oligonucleotide extension primer in the extension solution is extended by using the nucleic acid polymerizing enzyme to add a nucleotide analog to the oligonucleotide extension primer at the active site. This forms an extended oligonucleotide extension primer where the nucleotide analog being added is complementary to the nucleotide of the target nucleic acid molecule at the active site. The extension solution can then be passed through a molecular weight filter to separate the amplified target nucleic acid molecule and the extended oligonucleotide extension primer from the low molecular weight reaction components. The nucleotide analog added to the oligonucleotide extension primer at the active site is then determined by electrospraying the extended oligonucleotide extension primer.

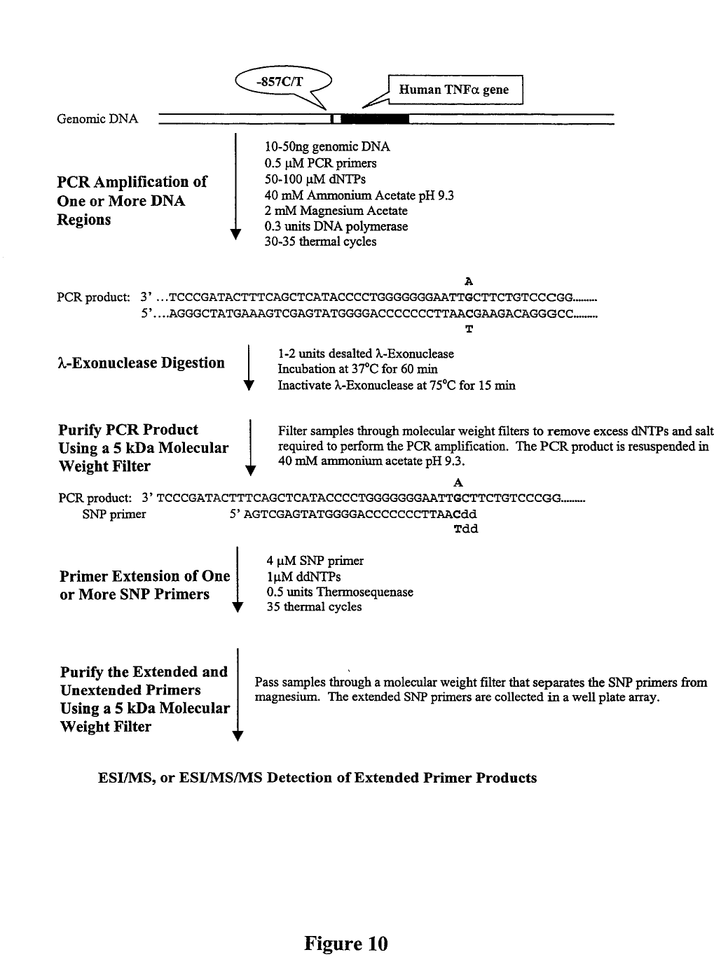

FIG. 10 shows the overall scheme of this embodiment of the present invention. Immediately following the PCR amplification, a λ-exonuclease digestion is performed using 1 to 2 units of desalted enzyme. The reaction is incubated for 1 hour at 37° C. During this incubation period, the enzyme converts the double-stranded PCR product into single-strand DNA, by progressively cleaving mononucleotides starting at the 5′-phosphorylation site. The enzyme is then inactivated by heating the reaction at 75° C. for 15 min. Then, instead of treating the reaction solution with a phosphatase, it is filtered through a molecular weight filter designed to retain the amplified DNA and the amplification primer, and allow the unreacted dNTPs to pass through. The amplified DNA is washed sufficiently with buffer solution to adequately remove the amplification primers and dNTPs. Following the washing step, the amplified DNA is resuspended in a buffer. Then, SNP primer, ddNTPs, and Thermosequenase® enzyme or other appropriate enzymes are combined and thermal cycled to perform the primer extension reaction. The extension solution can then be passed though a molecular weight filter to separate the amplified target nucleic acid molecule and the extended oligonucleotide extension primer from the low molecular weight reaction components. The nucleotide analog added to the oligonucleotide primer at the active site is then determined by electrospraying the extended oligonucleotide extension primer.

The ability to quantitatively determine the percent of minor allele component in the presence of a dominant allele in pooled DNA samples is a powerful means to streamline disease association and linkage studies. The present invention offers a means to perform these quantitative pooling studied in a one-well format. In addition, electrospray ionization is a more suitable ionization technique for quantitative studies than other techniques such as MALDI.

In a typical pooling quantitation study, both calibration curve standards and unknown samples would be analyzed. The calibration curve standards would consist of genomic DNA mixtures of the two possible homozygous samples, or one homozygous and one heterozygous. The ratios of the two homozygous samples would be made to encompass the working range of interest for quantifying the minor allele frequency. The unknown samples would consist of pooled genomic DNA samples from a population set. Then, one-well reactions in any of the formats discussed would be used to analyze both the calibration curve standards and the unknown samples. Following the analysis, the calibration curve standards would be used to make a calibration curve, or a graph of the percent consumption of the ddNTP complementary to the minor allele, which is determined experimentally, versus the percent of the minor allele in the standard, which was established at the time the calibration curve standards were made. The resulting calibration curve is then fit to an equation such as the Michaelis-Menten equation. After the equation of the calibration curve is determined and since the percent of the minor allele is experimentally determined, then the curve is used to establish the frequency of the minor allele in the pooled sample.

Typically, single-stranded DNA would be used in these quantitative pooling studies, since single-stranded template results in greater primer extension efficiency. Therefore, the one-well reaction used would most likely involve a λ-exonuclease digestion. However the quantitative pooling study would not be limited to one-well reactions. For example, following the PCR amplification and λ-exonuclease digestion, the calibration curve standards and the unknown samples, could be purified off-line using molecular weight filters to separate the PCR product from the low molecular weight reaction components. Then, the purified single-stranded DNA could be quantified, and used in the primer extension reactions.

In order to ensure that the calibration curve encompasses the concentration of range of the minor allele frequency of interest, several factors in the one-well reactions can be adjusted. These factors include the number of thermal cycles, the amount of Thermosequenase®, and the concentration of starting genomic DNA.

In carrying out the method of the present invention, genomic DNA can be extracted from whole blood, buccal epithelial cells, and saliva stain samples which are extracted by an alkaline method (Sweet et al., Forensic Sci. Int., 83:167-77 (1996); Lin et al., Biotechniques, 24:937-40 (1998); Rudbeck et al., Biotechniques, 25:588-90, 592 (1998), which are hereby incorporated by reference in their entirety). For blood, 5 μL of blood with 20 μL of 0.2 M NaOH are incubated at room temperature for 5 min. For an air-dried mouth swab, a proportion of the cotton is transferred to a tube, 20 μL of 0.2 M NaOH are added, and incubation is carried out at 75° C. for 10 min. This extraction procedure is carried out by adding 180 μL of 0.04 M Tris-HCl, pH 7.5. 5 μL of the above solution is sufficient for a subsequent 50 μL PCR reaction.

PCR amplification, a potential phosphatase digestion of unreacted dNTPs, a potential exonuclease I digestion, a potential λ-exonuclease digestion, and primer extension step are consecutively performed in the same well plate followed by electrospray mass spectrometry detection of the SNP bases.

The electrospray/mass spectrometery procedure is carried out so that the samples are analyzed in the negative ion mode. Selected reaction monitoring (“SRM”) mass spectrometry/mass spectrometry (“MS/MS”) experiments monitor unique precursor-product ion transitions for each ddNTP. For ddCTP, the SRM transition is either m/z 450→m/z 159 or m/z 370→m/z 79. For ddTTP, the SRM transition is either m/z 465→m/z 159 or m/z 385→m/z 79. For ddATP, the SRM transition is either m/z 474→m/z 159 or m/z 394→m/z 79. For ddGTP, the SRM transition is either m/z 490→m/z 159 or m/z 410→m/z 79. The relative concentration of the ddNTPs in each sample is compared to a non-extended reaction control. The base(s) complementary to the consumed ddNTPs during the primer extension reaction can be assigned as the SNP base for both homozygous and heterozygous alleles based upon the relative ion responses of each of the four ddNTPs.

The process of the present invention can be used to determine the single nucleotide variations of any target nucleic acid molecule, including RNA, double-stranded or single-stranded DNA, single-stranded DNA hairpins, DNA/RNA hybrids, RNA with a recognition site for binding of the polymerase, or RNA hairpins.

Nucleotide analogs which are useful in carrying out the present invention by serving as substrate molecules for the nucleic acid polymerizing enzyme include dNTPs, NTPs, modified dNTPs or NTPs, peptide nucleotides, modified peptide nucleotides, or modified phosphate-sugar backbone nucleotides.

The oligonucleotide extension primer used in carrying out the process of the present invention can be a ribonucleotide, deoxyribonucleotide, modified ribonucleotide, modified deoxyribonucleotide, peptide nucleic acid, modified peptide nucleic acid, modified phosphate-sugar backbone oligonucleotide, and other nucleotide and oligonucleotide analogs. It can be either synthetic or produced naturally by primases, RNA polymerases, or other oligonucleotide synthesizing enzymes.

The nucleic acid polymerizing enzyme utilized in accordance with the present invention can be either DNA polymerases, RNA polymerases, or reverse transcriptases. Suitable polymerases are thermostable polymerases or thermally degradable polymerases. Examples of suitable thermostable polymerases include polymerases isolated from Thermus aquaticus, Thermus thermophilus, Pyrococcus woesei, Pyrococcus furiosus, Thermococcus litoralis, and Thermotoga maritima. Useful thermodegradable polymerases include E. coli DNA polymerase, the Klenow fragment of E. coli DNA polymerase, T4 DNA polymerase, T7 DNA polymerase, and others. Examples for other polymerizing enzymes that can be used to determine the sequence of nucleic acid molecules include E. coli, T7, T3, SP6 RNA polymerases and AMV, M-MLV and HIV reverse transcriptases. The polymerase can be bound to the primed target nucleic acid sequence at a primed single-stranded nucleic acid, a double-stranded nucleic acid, an origin of replication, a nick or gap in a double-stranded nucleic acid, a secondary structure in a single-stranded nucleic acid, a binding site created by an accessory protein, or a primed single-stranded nucleic acid.

The one-well assay of the present invention can be used with any SNP detection method that uses primer extension reactions such as fluorescence, immunoassay and mass spectrometry. Fluorescence detection may be used with the one well assay by several methods. The ddNTPs may be fluorescently labeled with fluorophores that fluoresce at wavelengths such that a unique fluorescent signal may be discriminated in the presence of the other three fluorescently labeled ddNTPs. The SNP primers may contain quencher molecules designed to absorb the fluorescent signal of the fluorescently-labeled ddNTPs that extend the SNP primer. Fluorescence detection of each well would indicate which bases were consumed in the reaction.

In one embodiment of the present invention, after primer extension and before electrospraying, the extension solution is prepared for mass spectral analysis by first passing the reaction solution though a metal chelating material, and then evaporating the effluent so that residual material is taken up in water. In order to maximize the amount of this residual material that dissolves in the water, the samples can be subjected to sonication. Sonication is carried out using a sonicator. Typically, sonication for a period of 5 to 10 minutes yields adequate sensitivity for mass spectral analysis.

Two types of metal chelating material were used to remove magnesium from the samples. One of the materials is an immobilized iminodiacetic acid gel which was purchased from the Pierce Chemical Company, Rockford, Ill. When using this material the samples were prepared for mass spectrometry analysis off-line. The second type of metal chelating material was made in-house by modifying the surface of a porous poly(vinylbenzyl chloride—divinylbenzene) (PVBC-DVB) monolith or a coated PVBC-DVB layer in a PEEK tube or microchip channel, creating a surface suitable for immobilized metal affinity chromatography (IMAC), as described in U.S. Provisional Patent Application Serial No. 60/269,973, filed on Feb. 20, 2001, which is hereby incorporated by reference in its entirety (FIG. 11A). To modify the PVBC-DVB surface, a capillary tube or microchip channel already containing the monolith is filled with a degassed solution of 20% (v/v) diethyl iminodiacetate (DIDA) in acetonitrile. The tube or channel is then sealed and heated at 80° C. for 24 hours. Following the heat treatment, the DIDA solution is removed from the tube or channel before it is washed with acetonitrile and water. Next, the tube or channel is filled with 1 M NaOH and heated at 80° C. for 16 hours. Finally, the 1 M NaOH is removed from the tube or channel and it is washed with water, methanol, 0.1 M HCl, and water in that order. When using this material, the samples were prepared for mass spectrometry analysis on-line as shown in FIG. 11B.

Electrospray ionization provides for the atmospheric pressure ionization of a liquid sample (Kebaril et al., Electrospray Ionization Mass Spectrometry, 3-63 (1997), which is hereby incorporated by reference in its entirety). The electrospray process creates highly-charged droplets that, under evaporation, create ions representative of the species contained in the solution. When a positive voltage is applied to the tip of the capillary relative to an extracting electrode such as one provided at the ion-sampling orifice of a mass spectrometer, the electric field causes positively-charged ions in the fluid to migrate to the surface of the fluid at the tip of the capillary. If a negative voltage is applied to the tip of the capillary relative to an extracting electrode such as one provided at the ion-sampling orifice to the mass spectrometer, the electric field causes negatively-charged ions in the fluid to migrate to the surface of the fluid at the tip of the capillary.

When the repulsion force of the solvated ions exceeds the surface tension of the fluid being electrosprayed, a volume of the fluid is pulled into the shape of a cone, known as a Taylor cone, which extends from the tip of the capillary. A liquid jet extends from the tip of the Taylor cone and becomes unstable and generates charged-droplets. These small charged droplets are drawn toward the extracting electrode. The small droplets are highly-charged and solvent evaporation from the droplets results in the excess charge in the droplet residing on the analyte molecules in the electrosprayed fluid. The charged molecules or ions are drawn through the ion-sampling orifice of the mass spectrometer for mass analysis. This phenomenon has been described, for example, by Dole et al., Chem. Phys., 49:2240 (1968) and Yamashita et al., J. Phys. Chem., 88:4451 (1984), which are hereby incorporated by reference in their entirety. The potential voltage required to initiate an electrospray is dependent on the surface tension of the solution as described by, for example, Smith, IEEE Trans. Ind. Appl., IA-22:527-35 (1986), which is hereby incorporated by reference in its entirety. Typically, the electric field is on the order of approximately 106 V/m. The physical size of the capillary and the fluid surface tension determines the density of electric field lines necessary to initiate electrospray. Cole, Electrospray Ionization Mass Spectrometry: Fundamentals, Instrumentation, and Applications, (1997), which is hereby incorporated by reference in its entirety, summarizes much of the fundamental studies of electrospray. Several mathematical models have been generated to explain the principals governing electrospray.

U.S. patent application Ser. Nos. 09/468,535, 09/156,507, 09/764,698, 09/878,495, and 09/748,518, which are hereby incorporated by reference in their entirety, disclose suitable electrospray devices as well as methods and systems of using electrospray devices to prepare a sample for mass spectrometry.

The electrospray device used in conjunction with the present invention includes a substrate having an injection surface and an ejection surface opposing the injection surface. The substrate is an integral monolith having one or more spray units for spraying the fluid. Each spray unit includes an entrance orifice on the injection surface, an exit orifice on the ejection surface, a channel extending between the entrance orifice and the exit orifice, and a recess extending into the ejection surface and surrounding the exit orifice to define a nozzle on the ejection surface. The entrance orifices for each spray unit are in fluid communication with one another, and each spray unit generates an electrospray of the fluid. The electrospray device also includes a first electrode attached to the substrate to impart a first potential to the substrate and a second electrode to impart a second potential. The first and the second electrodes are positioned to define an electric field surrounding the exit orifice.

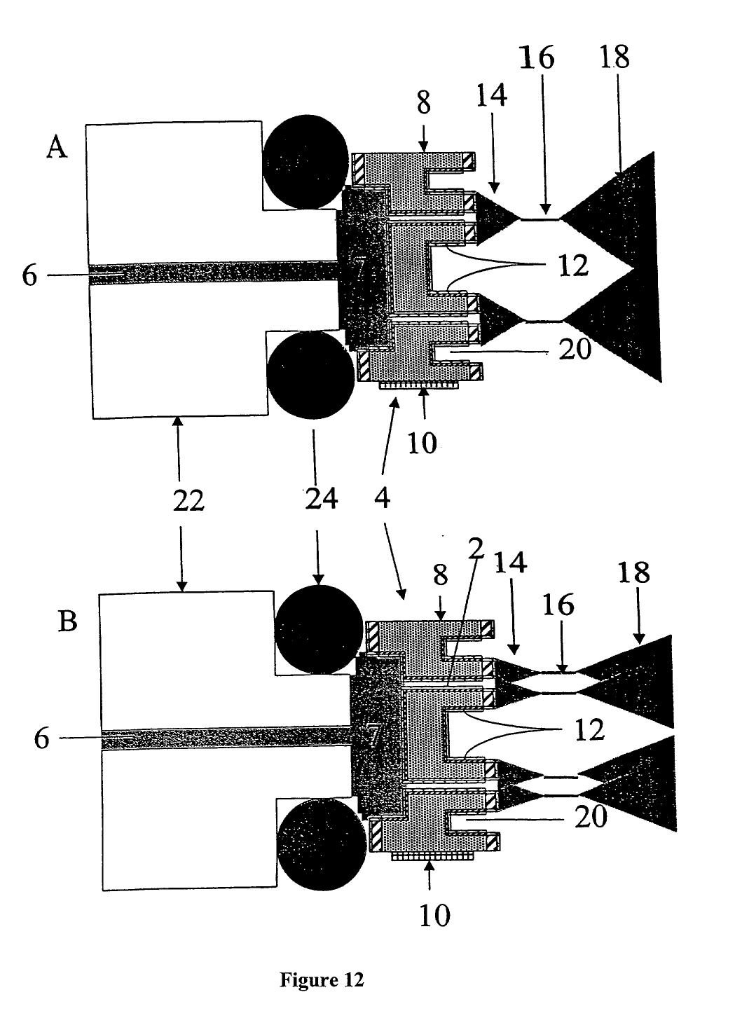

As shown in FIGS. 12A-B, to generate an electrospray, fluid may be delivered to the through-substrate channel 2 of the electrospray device 4 by, for example, a capillary 6, micropipette or microchip 22. Seal 24 is positioned between the microchip 22 and the electrospray device 4. The fluid is subjected to a potential voltage in the capillary 6, in the reservoir 7, or via an electrode provided on the reservoir surface and isolated from the surrounding surface region and the substrate 8. A potential voltage may also be applied to the silicon substrate via the electrode 10 on the edge of the silicon substrate 8, the magnitude of which is preferably adjustable for optimization of the electrospray characteristics. The fluid flows through the channel 2 and exits from the nozzle 12 in the form of a Taylor cone 14, liquid jet 16, and very fine, highly charged fluidic droplets 18.

The nozzle 12 provides the physical asperity to promote the formation of a Taylor cone 14 and efficient electrospray 18 of a fluid. The nozzle 12 also forms a continuation of, and serves as an exit orifice of, the through-wafer channel 2. The recessed annular region 20 serves to physically isolate the nozzle 12 from the surface. The present invention allows the optimization of the electric field lines emanating from the fluid exiting the nozzle 12 through independent control of the potential voltage of the fluid and the potential voltage of the substrate 8.

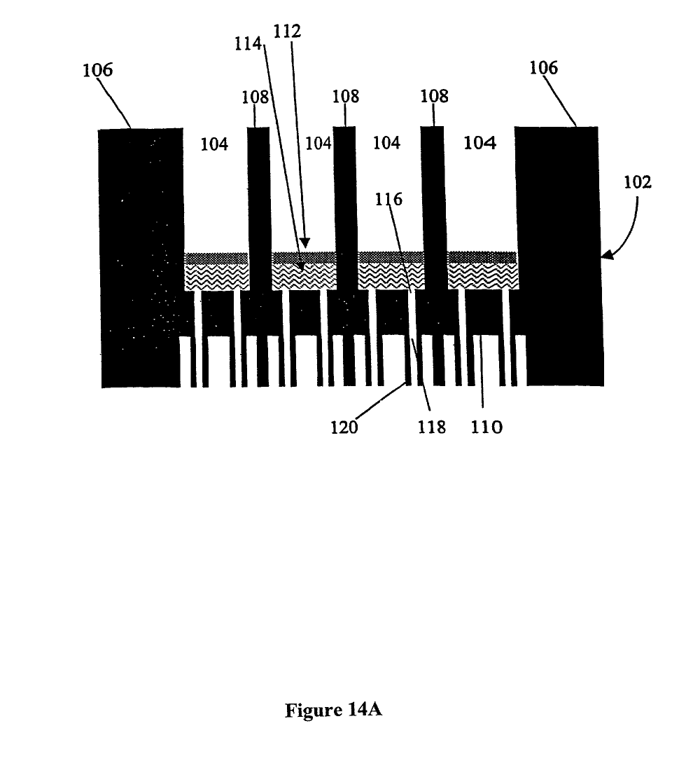

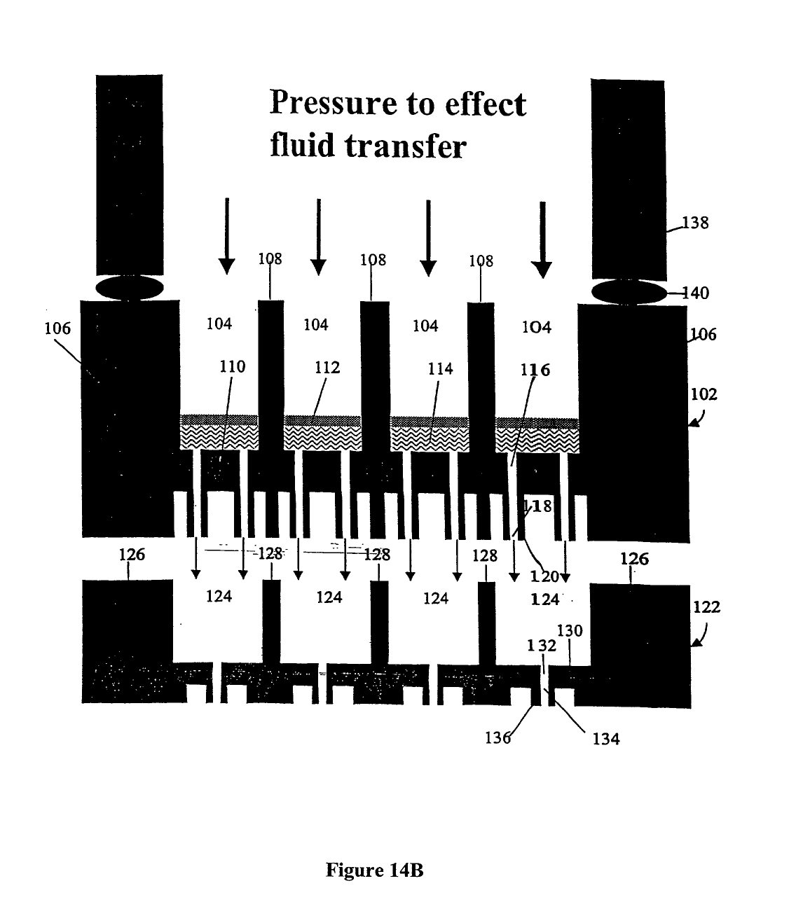

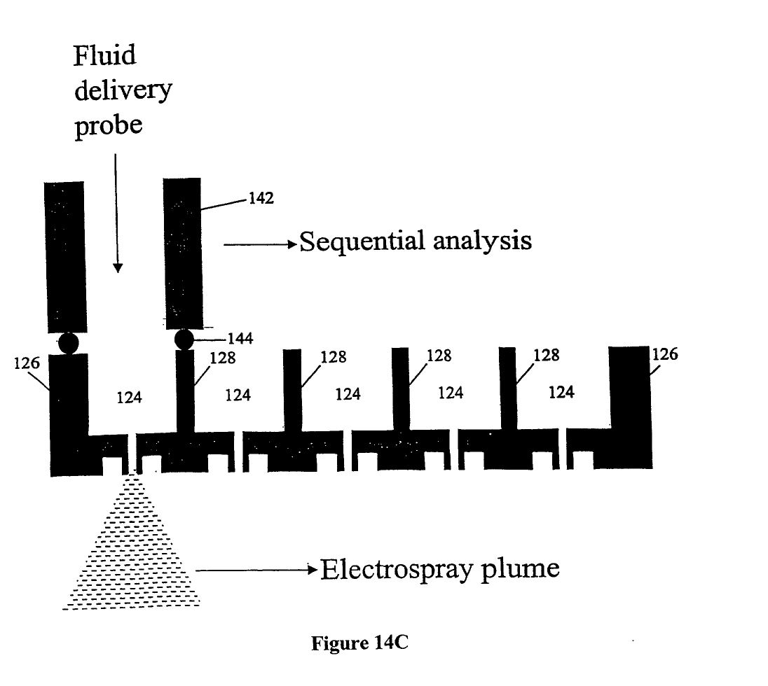

The system can be used with an array of reaction wells, preferably of volume less than 10 μL. The array is preferably in the same layout and spacing of standard 96, 384, 1536, and 6,144 well plates, although any array is suitable and may be optimized for a given application. As shown in FIGS. 13 and 14, the top layer consists of a reaction well which is where PCR amplifications, any digestion reactions, and primer extension reactions would be performed. The middle layer has a sample cleanup phase, preferably a metal chelating material, for the removal of magnesium from the reaction mixture. Also, a frit and a molecular weight filter may be used either by itself or together with a metal chelating material. The bottom layer has receiving wells in fluid communication with nozzles contained on a microchip for generating an electrospray of the reaction well product solution.

A reaction well block can be used for performing reactions, such as polymerase chain reactions and primer extensions. As shown in FIG. 14A, this aspect of the present invention is in the form of an array 102 of reaction wells 104, formed between plate edges 106 and/of walls 108. Wells 104, proximate to base 110, contain frit 112 or other medium separating the solution from the metal chelating resin. Liquid is discharged from wells 104 into entrance orifice 116, through channel 118 and out of exit orifice 120.

The system incorporates reaction wells with volumes on the order of tens of microliters to less than a microliter. The present invention has several advantages over other systems disclosed in the prior art. The double-stranded amplified target DNA fragment can be added directly to the reaction well array without prior separation of the strands. The SNP primers can be free in solution, thus increasing the reaction probability with the target DNA during the primer extension thermal cycles. The SNP primer used for each reaction is also an excess reagent relative to the added amount of each of the ddNTPs, thus effectively improving the incorporation efficiency (rate) of the target dideoxynucleotide base(s). The ddNTPs are added as a limiting reagent so that the ddNTPs that react and extend the SNP primer will be substantially consumed from the reaction solution. The reaction solution is then passed through a metal chelating material either on- or off-line to prepare the solution for electrospray mass spectrometry analysis. The relative response of the four ddNTP bases identifies by which base(s) the SNP primer was extended. Heterozygous SNPs can be identified if two ddNTP bases react with the SNP primer. In addition, this method can be used for discovery of the known point variation with both tri-allelic and tetra-allelic SNPs.

The electrospray system also includes a sample preparation device, as shown in FIG. 14A, positioned to transfer fluids to the electrospray device where the sample preparation device contains a liquid passage and a metal chelating material positioned to treat fluids passing through the liquid passage. Instead of a metal chelating agent, the sample preparation device can have a molecular weight filter positioned to treat fluids passing through the liquid passage. Alternatively, the sample preparation device could contain both a metal chelating material and a molecular weight filter.

This electrospray system is shown in FIG. 14B and includes an array 102 of reaction wells 104 each positioned to discharge liquid into a electrospray microchip 122. In particular, each exit orifice 120 is positioned to discharge liquid into a particular receiving well 124 which is formed between edges 126 and/or walls 128. After making this transfer, solutions evaporate in receiving wells 124 to dryness and are subsequently hydrated for controlled discharge. Liquid is discharged from the receiving well 124 through the base 130, via the entrance orifice 132, channel 134, and exit orifice 136. As a result, liquid is discharged from electrospray microchip 122 as an electrospray. Preferably, the electrospray microchip 122 is positioned in front of an ion-sampling orifice of an atmospheric pressure ionization mass spectrometer for analysis of the ddNTPs.

Another embodiment of the present invention would interface a microchip-based array of separation channels for the detection of ddNTPs with the reaction well array. The ddNTPs may be separated by liquid chromatography or electrophoretic methods, and quantified using spectroscopic or conductometric detection. A multi-system chip can be fabricated using Micro-ElectroMechanical System (MEMS) technology (Schultz et al., Anal. Chem., 72:4058-63 (2000), which is hereby incorporated by reference in its entirety) to further provide a rapid sequential chemical analysis system for large-scale SNP genotyping. For example, the multi-system chip enables automated, sequential separation and injection of a multiplicity of samples, resulting in significantly greater analysis throughput and utilization of the mass spectrometer instrument for high-throughput SNP detection.

As shown in FIG. 14B, liquid is fed into the entire depicted array 102 of reaction wells 104 through a conduit 132. A seal 140 is positioned between the edge 106 and the conduit 138 to prevent leakage. In addition, as shown FIG. 14C, a fluid delivery probe 142 is positioned against edges 126 and/or walls 128 by means of the seal 144 to permit liquid to be charged to the individual receiving wells 124. After each receiving well is filled, a probe 142 can move sequentially to the next well and fill it.

Due to its sensitivity and specificity with regard to low molecular weight entities, mass spectrometry is preferably used for the detection of these four ddNTPs independent of the SNP under evaluation. The mass spectrometry instrument and detection method is setup to screen any SNP by monitoring four unique ion response channels, one for each ddNTP. By use of nanomolar detection sensitivity, the electrospray mass spectrometry method is able to provide a rapid, selective, and sensitive method for SNP screening.

### EXAMPLES

The following examples are provided to illustrate embodiments of the present invention but are by no means intended to limit its scope.

### Example 1

### Preparation of a Polymer Monolithic IDA Column for On-Line Separation of Magnesium and ddNTPs

In order to create a surface suitable for immobilized metal affinity chromatography (IMAC), a procedure for the surface modification of a porous poly(vinylbenzyl chloride—divinylbenzene) (poly(VBC-DVB)) monolith or a coated PVBC-DVB layer in a PEEK tube or microchip channel can be performed. To modify the poly(VBC-DVB) surface, a capillary tube or microchip channel already containing the monolith is filled with a degassed solution of 20% (v/v) diethyl iminodiacetate (DIDA) in acetonitrile. The tube or channel is then sealed and heated at 80° C. for 24 hours. Following the heat treatment, the DIDA solution is removed from the tube or channel before it is washed with acetonitrile and water. Next, the tube or channel is filled with 1 M NaOH and heated at 80° C. for 16 hours. Finally, the 1 M NaOH is removed from the tube or channel, by washing with water, methanol, 0.1 M HCl, and water in such order.

FIG. 11A shows the scanning electron micrograph of a poly(VBC-DVB) monolith with IDA groups grafted to the polymer surface protruding from a PEEK capillary with a 500 μm inner diameter. A 4 cm length of the monolith IDA column was cut and placed in-line between a Perkin-Elmer Series 200 autosampler (Norwalk, Conn.) and a Micromass Quattro II triple quadrupole mass spectrometer (Cheshire, UK), as shown in FIG. 11B. Following the primer extension reaction, samples from a 96-well plate were injected onto the monolith metal chelating column for on-line separation of magnesium and ddNTPs prior to ESI/MS/MS analysis. The mobile phase consisted of 50% methanol in water with 0.1% acetic acid. The monolith was washed with mobile phase for 2 to 3 minutes between injections.

### Example 2

### A Preliminary One-Well SNP Assay for Human Genomic DNA Samples

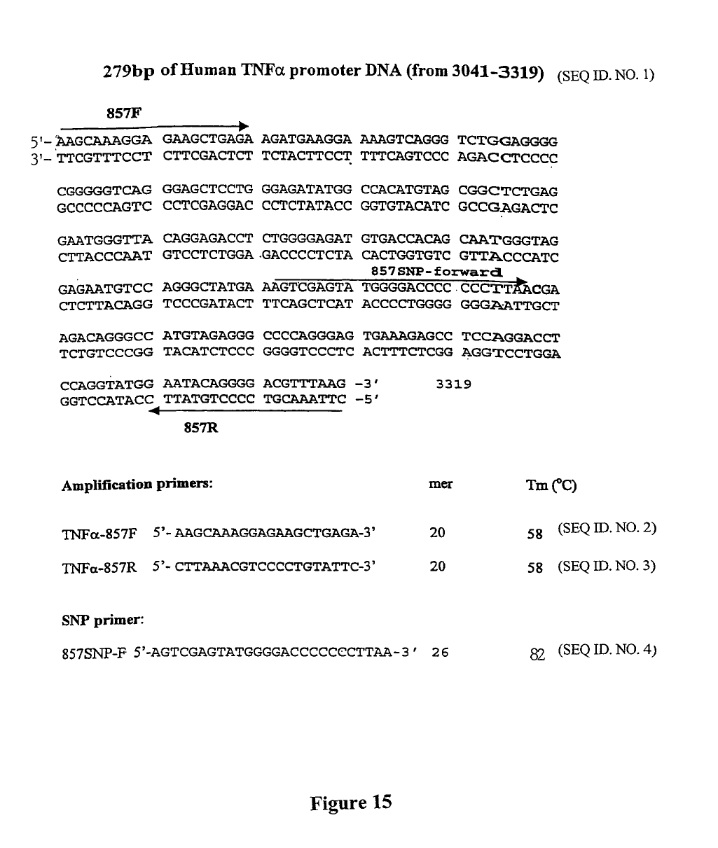

The 279 bp DNA sequence of human TNFα gene promoter region (SEQ. ID. No. 1; the complementary strand also shown) and the primers used as a model system for the present invention are shown in FIG. 15. Both amplification primers TNFα-857F (SEQ. ID. No. 2) and TNFα-857R (SEQ. ID. No. 3) consist of a 20mer with Tₘ=58° C. while the SNP primer 857SNP-F (SEQ. ID. No. 4) is a 26mer with Tₘ=82° C. The genomic DNA sample numbered NA03580-HD50G was purchased from Coriell Cell Repositories (Camden, N.J.) and was determined to be a heterozygous sample with genotype CT at position −857 of the TNFα gene promoter region.

The PCR amplification reaction was set up in a total volume of 10 μL consisting of 40 mM ammonium acetate pH 9.3, 50 μM dNTPs, 0.5 μM amplification primers, 2 mM magnesium acetate, 0.3 units of exo⁻ pfu DNA polymerase and 15 ng of human genomic DNA. The amplification was performed in a GeneAmp PCR System 9700 (Applied Biosystems, Foster City, Calif.) for 35 cycles with each cycle composed of 95° C. for 60 sec, 50° C. for 30 sec, and 72° C. for 60 sec, followed by an additional extension step at 72° C. for 5 minutes. Following amplification, one unit of dialyzed calf intestinal alkaline phosphatase (CIP) was added to each reaction and subsequently incubated at 37° C. for 60 min followed by 15 minutes at 75° C. for inactivation of CIP. Then, to the reaction mixture was added 1 μM ddNTPs, as well as 4 μM SNP primer, and 0.5 units of Thermosequenase®. For the control sample, water was added to replace SNP primer. The resultant mixture was subjected to 40 thermal cycles with each cycle containing 95° C. for 30 sec, 65° C. for 30 sec, and 72° C. for 30 sec. The final reaction samples were then passed through a micro-metal chelating column composed of immobilized iminodiacetic acid gel from Pierce Chemical Company (Rockford, Ill.) as reported previously (Zhang et al., Anal. Chem., 73:2117-25 (2001), which is hereby incorporated by reference in its entirety). The resulting samples were analyzed by electrospray ionization coupled to a Micromass triple quadrupole Quattro II mass spectrometer. A mobile phase composition of 1:1 methanol:water with 0.1% acetic acid was used at a flow rate of 150 μL/min. 10 μL injections were made for each sample via flow injection analysis. The mass spectrometer was equipped with a Z-spray source and operated in negative ion MS/MS selected reaction monitoring (SRM) mode. The Z-spray desolvation temperature and capillary voltage were 400° C. and 3000 V, respectively. The collision energy used was 35 V and the dwell time for each transition was 50 msec. The following selected reaction monitoring (SRM) transitions were monitored for each of the bases: ddCTP, m/z 370.1→m/z 79.0; ddTTP, m/z 385.1→m/z 79.0; ddATP, m/z 394.1→m/z 79.0; ddGTP, m/z 410.1→m/z 79.0. The relative concentration and ratio of each ddNTP in test samples were compared to those of a non-extended reaction control. The base(s) complementary to the consumed ddNTPs during the primer extension reaction can be assigned as the SNP base(s) for both homozygous and heterozygous alleles on the basis of the relative ion responses for each of the four ddNTPs. Furthermore, by mathematically normalizing the area ratios for the samples to those of the control, the percent of ddNTPs remaining in solution after reaction can be calculated.

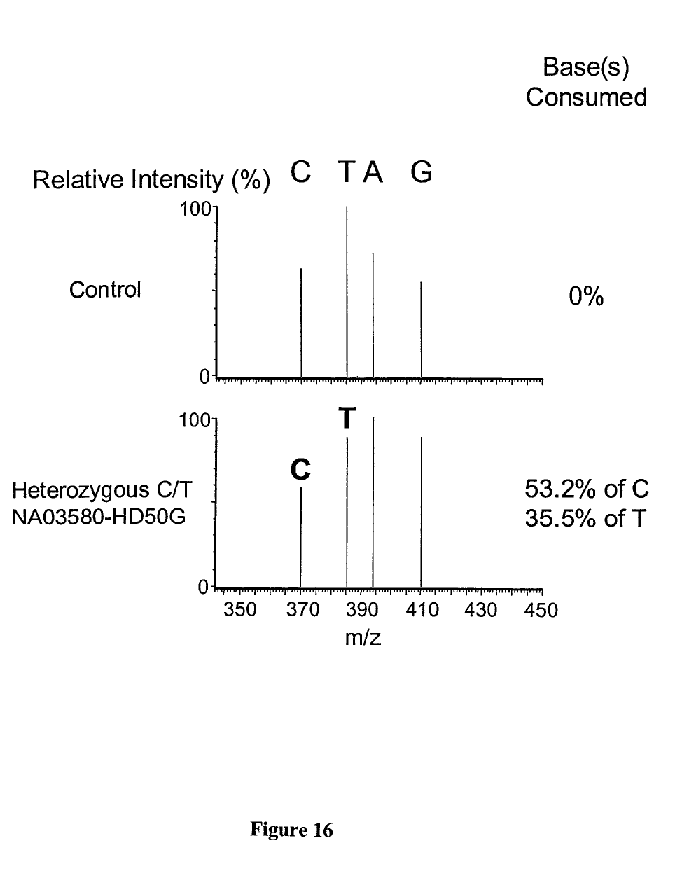

The SRM ESI/MS/MS initial results using genomic DNA samples in a one-well assay are shown in FIG. 16 and the relative peak ratios for detected ddNTPs are shown in Table 1. Top and bottom panels of FIG. 16 correspond to a control sample and a test sample NA03580-HD5OG, respectively. The mass spectra shown in FIG. 16 indicate that the intensity of the ions corresponding to both ddCTP and ddTTP are significantly lower than the intensity of those ions in the control. The test sample was known to be heterozygous CT determined by the same SNP primer in non-one well assay (Zhang et al., Anal. Chem., 73:2117-25 (2001), which is hereby incorporated by reference in its entirety). The present one-well results are consistent with that expected as a heterozygous CT at −857 of human TNFα gene promoter region. The target ddCTP was consumed 53.2% while ddTTP was consumed 35.4% during the primer extension step. The results demonstrate the feasibility of a one-well assay used for the SNP analysis.

TABLE 1
Peak Area Ratios of the ddNTPs Remaining in Solution
Following a Primer Extension Reaction for the Samples
whose Mass spectra are Shown in FIG. 16

| | Peak Area Ratios* | | | | | |
|---|---|---|---|---|---|---|
| | 370/385 | 370/394 | 370/410 | 385/394 | 385/410 | 394/41 |
| Sample | C/T | C/A | C/G | T/A | T/G | A/G |
| No SNP Primer | 0.58 | 1.34 | 1.67 | 2.32 | 2.90 | 1.25 |
| Control | | | | | | |
| NA03580- | 0.42 | 0.65 | 0.75 | 1.55 | 1.80 | 1.16 |
| HD50G | | | | | | |
| Sample | | | | | | |

*Note:
370 denotes the transition m/z 370.1 → m/z 79.0
385 denotes the transition m/z 385.1 → m/z 79.0
394 denotes the transition m/z 394.1 → m/z 79.0
410 denotes the transition m/z 410.1 → m/z 79.0

### Example 3

### Validation of a One-Well Assay Using Double-Stranded DNA as Template and Development of On-Line Sample Preparation for High-Throughput Analysis of SNPs by ESI/MS/MS

A one-well reaction assay combining both PCR amplification and primer extension reaction steps and requiring no off-line purification of PCR product, was achieved by simple addition of reagent solution into a single well. Furthermore, on-line separation of magnesium and ddNTPS using an in-house made monolith metal chelating column required no off-line removal of magnesium from samples for MS analysis. This method eliminated the tedious and time-consuming steps of sample preparation, minimized sample handing, and offered a high-throughput analysis of SNPs by ESI/MS. The analysis time per sample was 2 minutes. The simplicity of this method has potential for full automation and parallel chromatography, and thus, reduced analysis time.

The assay was blindly validated with 6 SNPs in 5 different human genomic DNA regions for a total of 330 SNP samples. Five target regions referred to as A, B, C, D and E have lengths of 212 bp, 158 bp, 166 bp, 191 bp, and 251 bp, respectively. Each region contains one test SNP, except for region D which has two SNPs referred to as SNP 1 and SNP 2. The sequences of amplification primers and SNP primers for each region and SNP (SEQ. ID. Nos. 5-22) are listed in Table 2. The 55 genomic DNA samples from four Utah pedigree families #1333, #1340, #1341, and #1345 were purchased from Coriell Cell Repositories (Camden, N.J.) and used in the blind validation of the one-well assay and for testing the on-line sample preparation using a monolithic metal chelating column.

TABLE 2
Sequences of the Primers Used in the Validation of the One-Well Assay

| | | | |
|---|---|---|---|
| Primer | Sequence | Tm* | Length |

Region A

| | | | |
|---|---|---|---|
| Forward Amplification | 5′ GTTAACAATCAGCTTGCCAAAT 3′ | 60 | 22 |
| | (SEQ ID. NO. 5) | | |
| Reverse Amplification | 5′ CAGTTCTCCTCCACTGCCTTAT 3′ | 66 | 22 |
| | (SEQ ID. NO. 6) | | |
| Reverse SNP | 5′ GCAACTCATACCAGCCCATGGGTCTAC 3′ | 84 | 27 |
| | (SEQ ID. NO. 7) | | |

Region B

| | | | |
|---|---|---|---|
| Forward Amplification | 5′ ACCTTTTTCCATGTGGTAACTGA 3′ | 62 | 22 |
| | (SEQ ID. NO. 8) | | |
| Reverse Amplification | 5′ CACTAAATCAGCTTTAATCCCATT 3′ | 64 | 24 |
| | (SEQ ID. NO. 9) | | |
| Reverse SNP | 5′ GGACACTAAATCAGCTTTAATCCCATTATTAAGAAA 3′ | 94 | 36 |
| | (SEQ ID. NO. 10) | | |

Region C

| | | | |
|---|---|---|---|
| Forward Amplification | 5′ CTCCCCCATGTACTTCTTCGT 3′ | 64 | 21 |
| | (SEQ ID. NO. 11) | | |
| Reverse Amplification | 5′ GCAGATCATGGAGTCAAACACA 3′ | 64 | 22 |
| | (SEQ ID. NO. 12) | | |
| Reverse SNP | 5′ ACATTGTCAATGTGGCGCACAAAGGC 3′ | 78 | 26 |
| | (SEQ ID. NO. 13) | | |

Region D

| | | | |
|---|---|---|---|
| Forward Amplification | 5′ TTTGCCAACCCTTAAAATCAAT 3′ | 58 | 22 |
| | (SEQ ID. NO. 14) | | |
| Reverse Amplification | 5′ GCAGGACTTCAGTTCCACTGTT 3′ | 66 | 22 |
| | (SEQ ID. NO. 15) | | |
| Reverse SNP 1 | 5′ GCTATTTTTAGTCAGCCATGCATTTGGATTTTAC 3′ | 92 | 34 |
| | (SEQ ID. NO. 16) | | |
| Forward SNP 2 | 5′ GCAGCCCTGTCCAATGGAATACAACATT 3′ | 82 | 28 |
| | (SEQ ID. NO. 17) | | |

Region E

| | | | |
|---|---|---|---|
| Forward Amplification | 5′ TGTTTCTCTCCCATCCTCACTT 3′ | 64 | 22 |
| | (SEQ ID. NO. 18) | | |
| 5′ Pi-Forward Amplification | 5′ Pi-TGTTTCTCTCCCATCCTCACTT 3′ | 64 | 22 |
| | (SEQ ID. NO. 19) | | |
| Reverse Amplification | 5′ ACTTTGGTGGCTCGAGATTCTA 3′ | 64 | 22 |
| | (SEQ ID. NO. 20) | | |
| Forward SNP | 5′ CTCTCCCATCCTCACTTCCTCAACGC 3′ | 82 | 26 |
| | (SEQ ID. NO. 21) | | |
| Reverse SNP | 5′ CAGTCACCGCTCTGCCAGAACCGGGTC 3′ | 92 | 28 |
| | (SEQ ID. NO. 22) | | |

*Tm calculated using the equation: Tm = 2(A + T) +4(C + G)
In regions A, B, C, and E, one SNP was analyzed, and in region D, two SNPs were analyzed.
For each of the six SNPs, 55 human genomic DNA samples were analyzed.

The PCR amplification reaction was set up in a total volume of 10 μL consisting of 40 mM ammonium acetate pH 9.3, 50 μM dNTPs, 0.5 μM amplification primers, 2 mM magnesium acetate, 0.3 units of exo⁻ pfu DNA polymerase and 15 ng of human genomic DNA. The amplification was performed in a GeneAmp PCR System 9700 (Applied Biosystems, Foster City, Calif.) for 35-45 cycles with each cycle composed of 95° C. for 60 sec, 52° C. for 45 sec, and 72° C. for 45 sec followed by an additional extension step at 72° C. for 5 minutes. Following amplification, one unit of dialyzed calf intestinal alkaline phosphatase (CIP) was added to each reaction and subsequently incubated at 37° C. for 30-60 min followed by 15 minutes at 75° C. to inactivate the CIP. Then, to the reaction mixture was added 1 μM ddNTPs, as well as 4 μM SNP primer, and 0.5 units of Thermosequenase®. For the control sample, the SNP primer was omitted and water was substituted for SNP primer. The resultant mixture was subjected to 30-45 thermal cycles with each cycle consisting of 95° C. for 30 sec, 65-72° C. for 30 sec, and 72° C. for 30 sec.

After the primer extension reaction, the samples on the 96-well plate were injected using a Perkin-Elmer Series 200 autosampler onto an in-house-made poly(VBC-DVB) monolith metal chelating column (4 cm×500 μm I.D., as described in Example 1) for on-line separation of magnesium and ddNTPs prior to MS analysis. The mobile phase, consisting of 50% methanol in water with 0.1% acetic acid, was delivered at a flow rate of 150 μL/min. The monolith column was re-equilibrated with the mobile phase for 2-3 minutes between injections. The quantitative analysis of remaining ddNTPs was performed with a triple quadrupole Micromass Quattro II mass spectrometer with SRM transitions monitored for each of the ddNTP bases. Computer software processed the data comparing the peak area or height ratios of all four bases and compared the ratios obtained for the test samples to those of the control. The percent of ddNTPs remaining in solution after reaction or the percent of target ddNTPs consumed during the reaction were calculated.

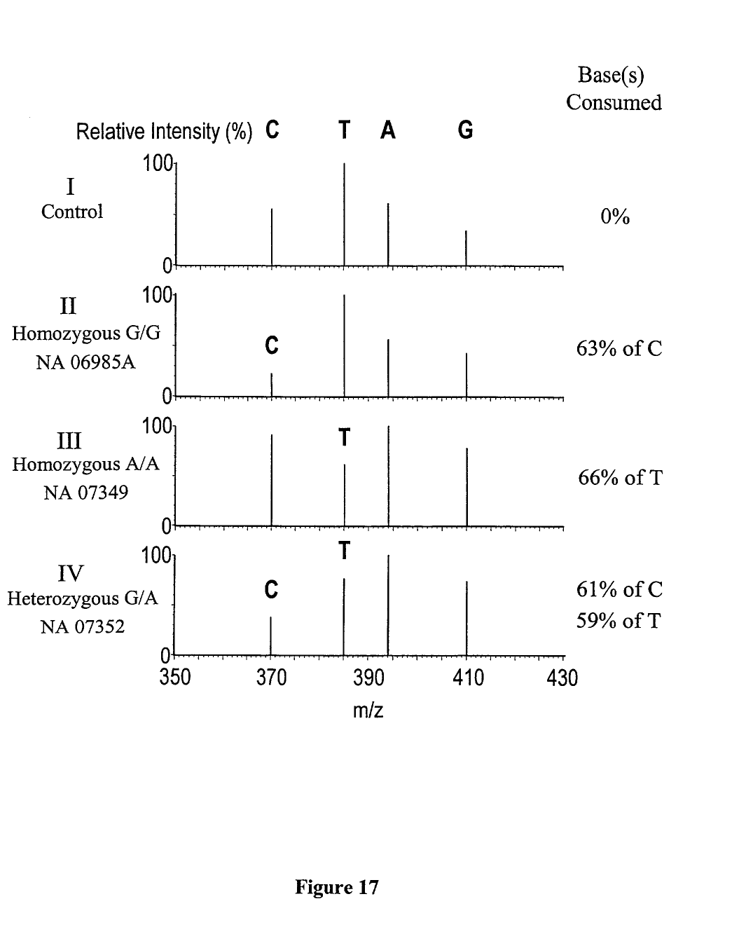

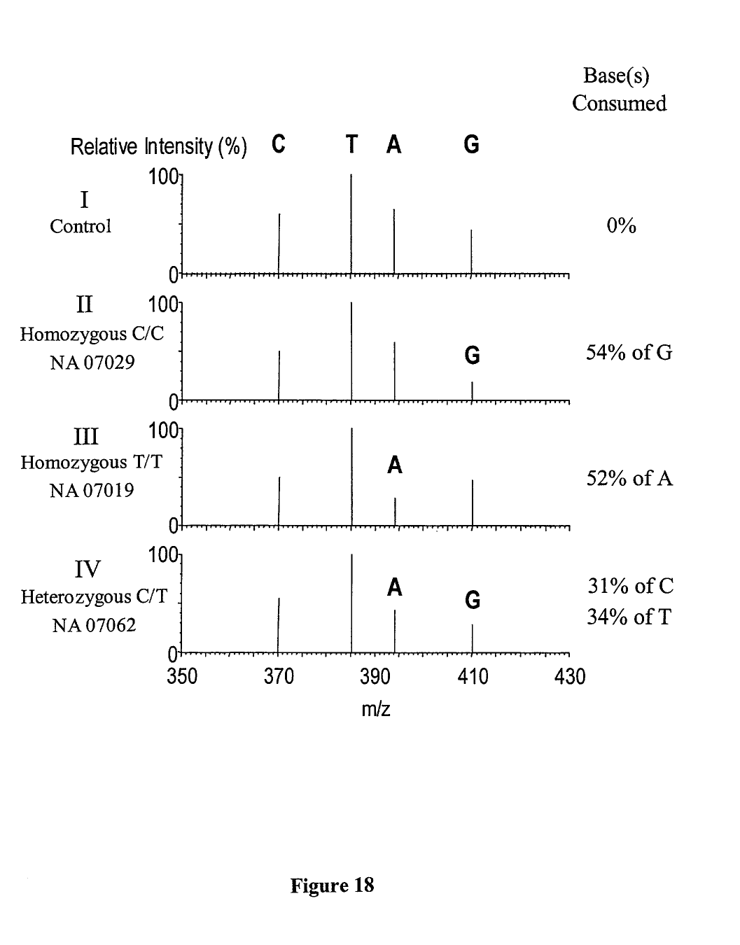

Two typical SRM ESI/MS/MS examples in this validation are shown in FIGS. 17 and 18, which correspond to regions B and D, respectively, and the relative peak ratios of the ddNTPs are shown in Tables 3 and 4, respectively. Panels I to IV of FIG. 17 correspond to a control sample, NA06985A, NA07349, and NA07352 samples, respectively. The mass spectra shown in FIG. 17, panels I-IV indicate that the intensity of the ions corresponding to ddCTP, ddTTP, and both ddCTP and ddTTP, respectively, are significantly lower than the intensity of those ions in the control. In both homozygous and heterozygous cases, the target ddNTPs were consumed about 60% during the primer extension step. Panels I to IV of FIG. 18 correspond to a control sample, NA07029, NA07019, and NA07062 samples respectively. The mass spectra shown in FIG. 18, panels I-IV indicate that the intensity of the ions corresponding to ddGTP, ddATP, and both ddGTP and ddATP, respectively, are significantly lower than the intensity of those ions in the control. In homozygous cases, the target ddNTPs were consumed above 50%, while in the heterozygous case, the target ddNTPs were consumed above 30% in the primer extension step.

TABLE 3
Peak Area Ratios of the ddNTPs Remaining in Solution
Following a Primer Extension Reaction for the Samples
whose Mass Spectra are Shown in FIG. 17

| | Peak Area Ratios | | | | | |
|---|---|---|---|---|---|---|
| | 370/385 | 370/394 | 370/410 | 385/394 | 385/410 | 394/410 |
| Sample | C/T | C/A | C/G | T/A | T/G | A/G |
| Control | 0.616 | 1.11 | 1.80 | 1.80 | 2.92 | 1.62 |
| Homozygous | 0.221 | 0.395 | 0.514 | 1.79 | 2.32 | 1.30 |
| G/G | | | | | | |
| NA 06985A | | | | | | |
| Homozygous | 1.50 | 0.907 | 1.17 | 0.605 | 0.777 | 1.28 |
| A/A | | | | | | |
| NA 07349 | | | | | | |
| Heterozygous | 0.492 | 0.383 | 0.522 | 0.777 | 0.106 | 1.36 |
| G/A | | | | | | |
| NA 07352 | | | | | | |

*Note:
370 denotes the transition m/z 370.1 → m/z 79.0
385 denotes the transition m/z 385.1 → m/z 79.0
394 denotes the transition m/z 394.1 → m/z 79.0
410 denotes the transition m/z 410.1 → m/z 79.0

TABLE 4
Peak Area Ratios of the ddNTPs Remaining in Solution
Following a Primer Extension Reaction for the Samples
whose Mass Spectra are Shown in FIG. 18

| | Peak Area Ratios | | | | | |
|---|---|---|---|---|---|---|
| | 370/385 | 370/394 | 370/410 | 385/394 | 385/410 | 394/410 |
| Sample | C/T | C/A | C/G | T/A | T/G | A/G |
| Control | 0.603 | 0.927 | 1.37 | 1.54 | 2.27 | 1.48 |
| Homozygous | 0.499 | 0.834 | 2.73 | 1.67 | 5.46 | 3.27 |
| C/C | | | | | | |
| NA 07029 | | | | | | |
| Homozygous | 0.497 | 1.76 | 1.07 | 3.55 | 2.16 | 0.608 |
| T/T | | | | | | |
| NA 07019 | | | | | | |
| Heterozygous | 0.546 | 1.28 | 1.96 | 2.35 | 3.59 | 1.53 |
| C/T | | | | | | |
| NA 07062 | | | | | | |

*Note:
370 denotes the transition m/z 370.1 → m/z 79.0
385 denotes the transition m/z 385.1 → m/z 79.0
394 denotes the transition m/z 394.1 → m/z 79.0
410 denotes the transition m/z 410.1 → m/z 79.0

The assay which used double-stranded DNA, was successfully validated with 5 SNPs in 4 different regions (Region A to D) for a total 275 SNP samples. The genotyping accuracy by this assay is 100% with all 275 samples correctly matching genotypes assigned by TaqMan and/or DNA sequencing as shown in Table 5. However, the SNP located in the 251 bp Region E failed to be genotyped using double-stranded DNA. Therefore, a modified one-well assay using single-stranded DNA was developed.

TABLE 5
Genotyping Results Obtained for Each Region Along with the Percent Accuracy

| | | | | Region D- | Region D- | |
|---|---|---|---|---|---|---|
| | Region A | Region B | Region C | SNP 1 | SNP 2 | Region E* |
| CEPH Family #01333 | | | | | | |
| NA07038A | GG | GG | CC | CC | CC | TT |
| NA06987A | GG | GA | CT | CC | CC | CC |
| NA07004 | GG | GA | CC | CC | CC | CT |
| NA07052 | GG | GG | CC | CC | CC | CT |
| NA06982 | GG | GA | CC | CC | CC | CT |
| NA07011 | GG | GA | CC | CC | CC | CT |
| NA07009 | GG | GA | CT | CC | CC | CT |
| NA07678A | GG | GG | CT | CC | CC | CT |
| NA07026 | GG | GA | CC | CC | CC | CT |
| NA07679 | GG | GA | CT | CC | CC | CT |
| NA07049 | GA | GG | CT | CC | CC | CT |
| NA07002 | GA | GA | CC | CT | CT | CT |
| NA07017 | GG | GG | CC | CT | CT | CC |
| NA07341 | GG | GA | CT | CT | CT | CC |
| NA11820 | GG | GG | CC | CC | CC | CT |
| CEPH Family #01340 | | | | | | |
| NA07029 | GA | GG | CC | CC | CC | CT |
| NA07019 | GA | GA | CC | TT | TT | CT |
| NA07062 | GA | GA | CC | CT | CT | CT |
| NA07053A | GG | GA | CC | CT | CT | CC |
| NA07008 | GG | GA | CC | CT | CT | TT |
| NA07040 | GG | GG | CC | CT | CT | CC |
| NA07342 | AA | GA | CC | CT | CT | CT |
| NA07027 | AA | GG | CC | CT | CT | CT |
| NA06994 | GG | GG | CC | CT | CT | TT |
| NA07000 | GA | GG | CC | CT | CT | CT |
| NA07022 | GA | GG | CC | CT | CT | CT |
| NA07056 | GA | GA | CC | CT | CT | CT |
| NA11821 | AA | GA | CC | CT | CT | CT |
| CEPH Family #01341 | | | | | | |
| NA07048 | AA | GG | CC | TT | TT | CC |
| NA06991 | GA | GG | CC | TT | TT | CT |
| NA07343A | AA | GG | CC | TT | TT | CT |
| NA07044 | AA | GG | CC | TT | TT | CC |
| NA07012 | AA | GG | CC | TT | TT | CC |
| NA07344 | AA | GG | CC | TT | TT | CC |
| NA07021 | GA | GG | CC | TT | TT | CT |
| NA07006 | GA | GG | CC | TT | TT | CC |
| NA07010A | GA | GG | CC | TT | TT | CT |
| NA07020 | GA | GG | CC | TT | TT | CC |
| NA07034A | GA | GA | CC | TT | TT | CC |
| NA07055A | GA | GG | CC | CT | CT | CC |
| NA06993 | GG | GG | CC | TT | TT | TT |
| NA06985A | GA | GG | CC | TT | TT | CT |
| CEPH Family #01345 | | | | | | |
| NA07349 | GG | AA | CC | CT | CT | CT |
| NA07348A | GG | GG | CC | CC | CC | CT |
| NA07350 | GG | GA | CC | CC | CC | CC |
| NA07351A | GG | GA | CC | CC | CC | CT |
| NA07352 | GG | GA | CC | CC | CC | CT |
| NA07353A | GG | GA | CC | CT | CT | TT |
| NA07354 | GG | GA | CC | CC | CC | CC |
| NA07355A | GG | GA | CC | CT | CT | CT |
| NA07356A | GG | GA | CC | CC | CC | TT |
| NA07347 | GG | GA | CC | CT | CT | CT |
| NA07346C | GG | GA | CC | CT | CT | CT |
| NA07357 | GG | GG | CC | CC | CC | CT |
| NA07345A | GG | GG | CC | CC | CC | CC |
| % Accuracy^{ξ} | 100 | 100 | 100 | 100 | 100 | 100 |

*The data was obtained by one-well assay using short (60nt) single-stranded template from region E.
^{ξ}Percent accuracy was obtained based on the TaqMan ™ assay and/or DNA sequencing.

### Example 4

### Validation of a One-Well Assay Using Single-Stranded DNA as Template Followed by On-Line Sample Preparation for High-Throughput Analysis of SNPs by ESI/MS/MS

As described in Example 3, the genotyping of the SNP in Region E, using the one-well assay, failed for all 55 genomic samples. Two SNP primers (SEQ. ID. Nos. 21 and 22) with two different directionalities were tested and gave rise to similar results. Thus, the strategy using λ-exonuclease for creating single-stranded DNA prior to CIP digestion and primer extension was tested. In addition, the efficiency of the primer extension in a one-well assay with single-stranded DNA was tested using two different lengths of amplified fragments (60 bp and 251 bp), both of which contained the same region E SNP.

The PCR amplification reaction for the 251 bp fragment of region E was set-up in a total volume of 10 μL. The reaction mixture consisted of 40 mM ammonium acetate pH 9.3, 50 μM dNTPs, 0.5 μM 5′ phosphorylated forward amplification primer (SEQ. ID. No. 19), 0.5 μM reverse amplification primer (SEQ. ID. No. 20), 2 mM magnesium acetate, 0.3 units of exo⁻ pfu DNA polymerase and 15 ng of human genomic DNA. For amplification of the 60 bp fragment of region E, the 10 μL reaction was composed of 40 mM ammonium acetate pH 9.3, 50 μM dNTPs, 0.5 μM 5′-phosphorylated forward amplification primer (SEQ. ID. No. 19), 0.5 μM reverse SNP primer (SEQ. ID. No. 22), 2 mM magnesium acetate, 0.3 units of exo⁻ pfu DNA polymerase and 15 ng of human genomic DNA. The amplification was performed in a GeneAmp PCR System 9700 (Applied Biosystems, Foster City, Calif.) for 35 cycles with each cycle composed of 95° C. for 60 sec, 50° C. for 30 sec, and 72° C. for 60 sec, followed by an additional extension step at 72° C. for 5 minutes.

Following amplification, one unit of dialyzed λ-exonuclease was added to the reactions and they were incubated at 37° C. for 60 min, allowing the enzyme to progressively cleave 5′-mononucleotides from the phosphorylated strand of PCR product. And then one unit of dialyzed CIP was added to each reaction and subsequently incubated at 37° C. for 60 min followed by 15 minutes at 75° C. for inactivation of both λ-exonuclease and CIP. To the reaction mixture was added 1 μM ddNTPs, as well as 4 μM forward SNP primer (SEQ. ID. No.21), and 0.5 units of Thermosequenase®. In the control sample, water was substituted for SNP primer. The resultant mixture was subjected to 30-45 thermal cycles with each cycle composed of 95° C. for 30 sec and 72° C. for 60 sec.

After the primer extension reaction, the samples in the 96-well plate were injected using a Perkin-Elmer Series 200 autosampler to an in-house-made poly(VBC-DVB) monolith metal chelating column (4 cm×500 μm I.D., as described in Example 1) for on-line separation of magnesium and ddNTPs prior to MS analysis. The mobile phase, consisting of 50% methanol in water with 0.1% acetic acid was delivered at a flow rate of 150 μL/min. The monolith column was re-equilibrated with the mobile phase for 2-3 minutes between injections. The quantitative analysis of unreacted ddNTPs was performed with a triple quadrupole Micromass Quattro II mass spectrometer with SRM transitions monitored for each of the ddNTP bases. Computer software processed the data comparing the peak area or height ratios of all four bases and compared the ratios obtained for the test sample to those of the control. The percent of ddNTPs remaining in solution after reaction or the percent of target ddNTPs consumed during the reaction were calculated.

Table 6 shows the comparison of percent consumption of ddCTP and ddTTP using long (251 bp) and short (60 bp) fragments of double-stranded and single-stranded DNA template for region E. The results indicate that using single-stranded DNA as template for region E in a one-well assay gave a very high efficiency of target ddNTPs incorporation in the primer extension, however the double-stranded template of region E failed to give more than 30% consumption. Only one CC homozygous in the short double-stranded template reached 35.8% consumption. In both single-stranded DNA homozygous cases the target ddNTPs were consumed about 70% during the primer extension step, while in the heterozygous case, there is a significant difference between the long and short templates. Under the same conditions, when long single-stranded template was used in the analysis of the heterozygous sample, the target ddNTPs were consumed by 40-50% while when the short single-stranded template was used target ddNTPs were consumed by 60-75%. The final results which are shown in column 7 of Table 5 were 100% accurate and were obtained using short single-stranded template in the one-well assay.

TABLE 6
Percent Consumptions of ddCTP and ddTTP for Long and Short,
Double-Stranded and Single-Stranded Template from Region E

| | Double-Stranded | | Double-Stranded | | Single-Stranded | | Single-Stranded | |
|---|---|---|---|---|---|---|---|---|
| | 251bp Template | | 60bp Template | | 251bp Template | | 60bp Template | |
| | % ddCTP | % ddTTP | % ddCTP | % ddTTP | % ddCTP | % ddTTP | % ddCTP | % ddTTP |
| Homozygous C/C | 17.2% | 11.3% | 35.8% | −1.6% | 78.0% | 9.20% | 70.8% | 0.606% |
| Homozygous T/T | −2.59% | −6.49% | 15.0% | 8.90% | −0.806% | 65.9% | 0.611% | 74.6% |
| Heterozygous C/T | 15.0% | 9.33% | 1.40% | −13.4% | 52.9% | 42.1% | 74.9% | 62.0% |

NOTE:
A genotype can be assigned only if a 30% or greater consumption of base is observed. Therefore, genotypes can be assigned only where the percent consumption values are bolded.

### Example 5

### Quantitating the Allele Frequency in a Model System of Pooled Synthesized Oligo Templates

Various combinations of synthesized oligo templates (SEQ. ID. No. 23-26) shown in Table 7, were pooled to investigate the possibility of quantitating the minor or mutant allele frequency in the presence of a dominant allele. The same SNP primer (SEQ. ID. No. 27), whose sequence is shown in Table 8, was used for all of the synthesized oligo templates. This model system allowed for many different combinations of minor and dominant alleles to be examined.

TABLE 7
Oligo Templates Used in the Quantitative Pooling Study

| Oligo Template | Sequence | |
|---|---|---|
| Oligo A | 5′ CCCCTGTATCCTGTGTGAAATTGTTATCCGCTC 3′ | (SEQ ID. NO. 23) |
| Oligo C | 5′ CCCCTGTCTCCTGTGTGAAATTGTTATCCGCTC 3′ | (SEQ ID. NO. 24) |
| Oligo G | 5′ CCCCTGTGTCCTGTGTGAAATTGTTATCCGCTC 3′ | (SEQ ID. NO. 25) |
| Oligo T | 5′ DDDDTGTTTCCTGTGTGAAATTGTTATCCGCTC 3′ | (SEQ ID. NO. 26) |

TABLE 8
Primers Used in the Quantitative Pooling Study

| | | | |
|---|---|---|---|
| Primer | Sequence | Tm* | Length |

For Oligo Templates

| | | | |
|---|---|---|---|
| #1233 | 5′ AGCGGATAACAATTTCACACAGGA 3′ | 68 | 24 |
| | (SEQ ID. NO. 27) | | |

Region C

| | | | |
|---|---|---|---|
| Forward Amplification | 5′ CTCCCCCATGTACTTCTTCGT 3′ | 64 | 21 |
| | (SEQ ID. NO. 11) | | |
| 5′ Pi-Reverse Amplification | 5′ Pi-GCAGATCATGGAGTCAAACACA 3′ | 64 | 22 |
| | (SEQ ID. NO. 28) | | |
| Reverse SNP | 5′ ACATTGTCAATGTGGCGCACAAAGGC 3′ | 78 | 26 |
| | (SEQ ID. NO. 13) | | |

*Tm calculated using the equation: Tm = 2(A + T) +4(C + G)

Primer extension reactions, which were composed of 40 mM ammonium acetate pH 9.3, 2 mM magnesium acetate, 1 μM of each of ddATP, ddCTP, ddGTP, and ddTTP, 4 μM SNP primer, 3 units of Thermosequenase®, and a total of 50 nM oligo template, were performed. The total concentration of oligo template was made up of the minor and dominant alleles. The percent of minor allele ranged from 0% to 100% of the total oligo template concentration. During the primer extension, the reactions were cycled either 10 or 60 times with each cycle composed of 30 sec at 95° C., 40 sec at 60° C., and 40 sec at 72° C. Following the primer extension reactions, the samples were prepared for MS analysis either on-line using an IDA polymer monolith column, or off-line by passing the reaction solution through an immobilized iminodiacetic acid gel obtained from Pierce Chemical Company (Rockford, Ill.), followed by evaporation of the effluent and reconstitution in water.

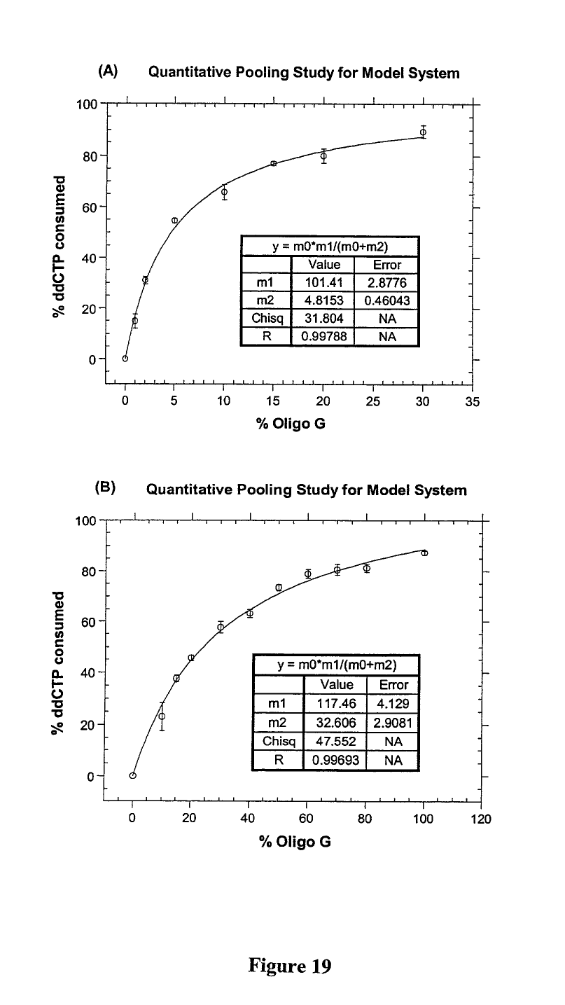

After the samples were analyzed, calibration curves for each minor allele and dominant allele variant were made. This was accomplished by first calculating the percent of ddNTP complementary to the minor allele consumed in the primer extension reaction by using the control sample to normalize what the ddNTP intensity would have been if no extension of the primer had occurred. Then, the calibration curve was made by plotting the percent of ddNTP consumed versus the percent of minor allele oligo present in the same reaction. The curves were fit to the Michaelis-Menten equation which is y=M₀M₁(M₀+M₂) where M₀ is the percent of minor allele, and both M₁ and M₂ are constants determined experimentally for each minor/dominant allele combination. FIG. 19 shows two calibration curves from quantitative pooling studies using synthesized oligos as templates. In both curves the minor or mutant allele was oligo G and the dominant allele was oligo A. FIG. 19A shows the calibration curve obtained when 60 thermal cycles were used, and the percent of oligo G in the primer extension reactions ranged from 0% to 30%. FIG. 19B shows the calibration curve obtained when 10 thermal cycles were used, and the percent of oligo G in the primer extension reactions ranged from 0% to 100%. Table 9 is a summary of the equations obtained for the different combinations of mutant and dominant alleles. All of these variations were obtained using 0% to 30% of the minor allele frequency and 60 thermal cycles. Also provided in Table 9 is the percent value of the mutant allele when 30% of the ddNTP is consumed. This value provides measure of the efficiency of the minor allele in the primer extension reactions.

TABLE 9
Summary of Pooling Study Results for Model Systems with
Different Combinations of Mutant/Dominant Alleles*

| | | | | | % Mutant Allele |
|---|---|---|---|---|---|
| Mutant Allele/ | Dominant | | | | when Y = 30% |
| Base Consumed | Allele | M₁^{ξ} | M₂ | R | consumption |
| Oligo G/ | Oligo A | 101.4 ± 2.9 | 4.8 ± 0.46 | 0.998 | 2.1 |
| ddCTP | | | | | |
| Oligo T/ | Oligo A | 105.9 ± 8.6 | 11.3 ± 2.19 | 0.994 | 4.5 |
| ddATP | | | | | |
| Oligo C/ | Oligo A | 95.8 ± 4.9 | 5.3 ± 0.82 | 0.995 | 2.4 |
| ddGTP | | | | | |
| Oligo A/ | Oligo G | 122.5 ± 15.6 | 12.4 ± 3.64 | 0.988 | 4.0 |
| ddTTP | | | | | |
| Oligo T/ | Oligo C | 90.5 ± 4.4 | 11.1 ± 1.28 | 0.998 | 5.5 |
| ddATP | | | | | |
| *The model system included a universal primer M13/pUC reverse sequence #1233 (5′ AGCGGATAACAATTTCACACAGGA 3′ (SEQ ID. NO. 27)) used as a SNP primer, and four 33mer synthetic target templates with the basic sequence 5′CCCCTGTNTCCTGTGTGAAATTGTTATCCGCTC 3′ | | | | | |
| #(basic template sequence of SEQ ID. NOs. 23-26). The four target sequences differed from one other only at the underlined polymorphic N site with an A, G, C or T base, and are named as Oligo A, G, C or T respectively. The sequence complementary to the #1233 primer is italicized in the target DNA sequence. All reactions were thermal cycled 60 times and contained a minor allele frequency in the range of 0% to 30%. | | | | | |

^{ξ}The constants for the Michaelis-Menten equation for each of the combinations are provided. All curves were fit to the equation: y = M₀M₁ / (M₀ + M₂) in which M₀ indicates the percent of mutant allele, while both M₁ and M₂ are characteristic constants experimentally obtained for each mutant/dominant allele combination under current conditions.

### Example 6

### Quantitating the Allele Frequency of Pooled, Purified, Single-Stranded DNA PCR Products from Region C

Region C from human genomic DNA samples were amplified and then converted to single-stranded DNA using λ-exonuclease digestions. Following the conversion, the single-stranded DNA was purified off-line using Microcon-50 filter units (Millipore, Bedford, Mass.) with a 50 kDa nominal molecular weight limit. The purified single-stranded DNA was then quantified spectrophotometrically. Finally, primer extension reactions were performed similar to those performed in Example 4.

Region C was amplified for a known homozygous C/C genomic DNA sample (NA 07038A), a known heterozygous C/T genomic DNA sample (NA 06987A), and three test samples, referred to as Pool 1, Pool 2, and Pool 3, respectively, which were made by pooling 15, 28, and 9 genomic DNA samples together, respectively. Pool 1 contains all 15 samples from family #01333. Pool 2 is a mixture of all samples from families #01333 and #01340. Pool 3 is composed of equal amounts of samples NA 07009, NA 07678A, NA 07026, NA 07679, NA 07049, NA 07002, NA 07017, NA 07341, and NA 11820 from family #01333. The 50 μL PCR amplification reactions consisted of 1× pfu buffer, 200 μM of each of dATP, dCTP, dGTP, and dTTP, 0.5 μM of forward amplification primer (SEQ. ID. No. 11), 0.5 μM of 5′-phosphorylated reverse amplification primer (SEQ. ID. No. 28), 1.25 units of pfu Turbo DNA polymerase, and 35 ng of genomic DNA. During the PCR amplification, the reactions were cycled 45 times with each cycle composed of 95° C. for 1 min, 52° C. for 45 sec, and 72° C. for 45 sec. Following the 45 cycles, the reactions were held at 72° C. for 5 min as an additional extension period. After the PCR amplification reaction, λ-exonuclease digestions were performed. To each 50 μL reaction were added 5.8 μL of 10× λ-exonuclease buffer, and 7.5 units of λ-exonuclease. The λ-exonuclease digestions were incubated at 37° C. for 60 min, before being heated at 75° C. for 15 min in order to inactivate the enzyme. A 2% agarose gel was run to verify that the double-stranded PCR product was converted into single-stranded DNA. Next, the single-stranded DNA samples were passed through Micropure-EZ membranes (Millipore, Bedford, Mass.) to remove proteins, and then purified with Microcon-50 filter units (Millipore, Bedford, Mass.) having a 50 kDa nominal molecular weight limit. These filter units isolated the single-stranded DNA from all of the small molecular weight reaction components. The single-stranded DNA was washed four times with 400 μL volumes of sterile water. Finally, the purified single-stranded DNA was quantified spectrophotometrically.

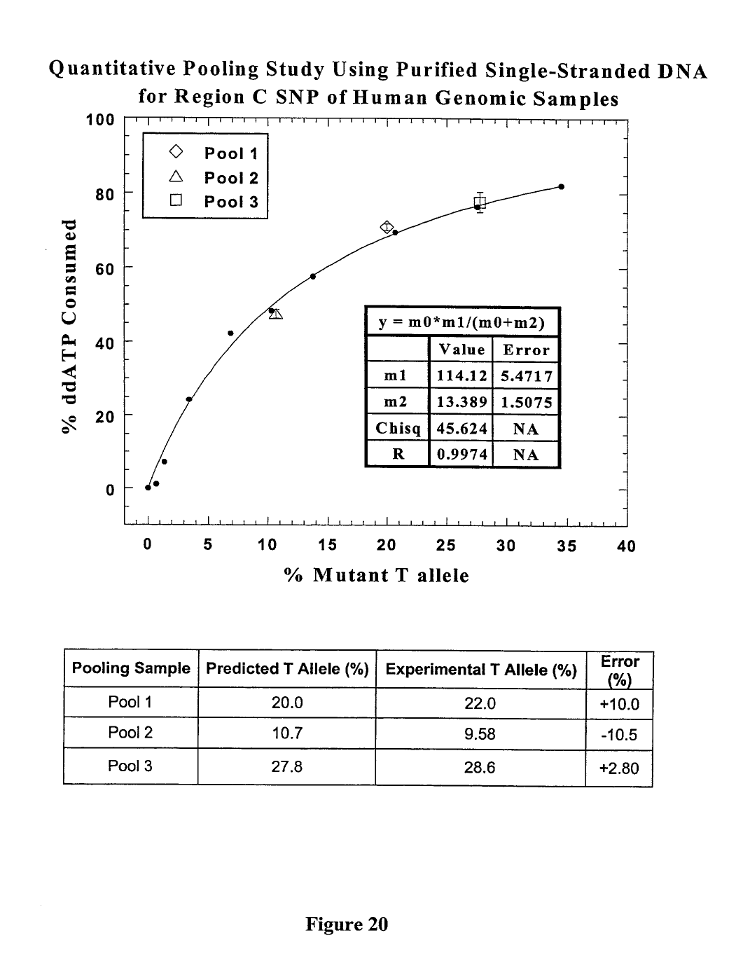

The primer extension reactions were composed of 40 mM ammonium acetate pH 9.3, 2 mM magnesium acetate, 1 μM of each of ddATP, ddCTP, ddGTP, and ddTTP, 5 μM SNP primer, 3 units of Thermosequenase®, and a total single-stranded DNA concentration of 50 nM. In the primer extension reactions, the calibration curve standards were made by combining purified single-stranded DNA from the homozygous C/C and the heterozygous C/T sample in different ratios so that the mutant T allele varied from 0% to 35% of the total template concentration. FIG. 20 shows the calibration curve and provides the Michaelis-Menten equation for the curve. The curve was constructed by the same means as described in Example 5. In addition, FIG. 20 also shows the results from the three test samples, Pool 1, Pool 2, and Pool 3, which were made by pooling genomic DNA samples together. The theoretical percent of the T allele was calculated to be 20.0%, 10.7%, and 27.8% for Pool 1, Pool 2, and Pool 3, while the experimental percent of the T allele was determined to be 22.0%, 9.58%, and 28.6%, so that the percent errors were +10.0%, -10.5%, and +2.80% respectively.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A method of detecting single nucleotide polymorphisms comprising:
providing a sample potentially containing a target nucleic acid molecule;
subjecting the sample to a polymerase chain reaction process involving use of oligonucleotide amplification primers under conditions effective to amplify any of the target nucleic acid molecule present in the sample to produce an amplification product;
subjecting the amplification product to treatment with a phosphatase under conditions effective to remove 5′ phosphates from free deoxynucleotide triphosphates (dNTPs) in the amplification product;
inactivating the phosphatase;
providing an oligonucleotide extension primer complementary to a portion of the target nucleic acid molecule;
providing a nucleic acid polymerizing enzyme;
providing a plurality of types of nucleotide analogs;
blending the amplification product treated with a phosphatase, the oligonucleotide extension primer, the nucleic acid polymerizing enzyme, and the nucleotide analogs, each type being present in a first amount, to form an extension solution where the oligonucleotide extension primer is hybridized to the target nucleic acid molecule to form a primed target nucleic acid molecule and the nucleic acid polymerizing enzyme is positioned to add nucleotide analogs to the primed target nucleic acid molecule at an active site;
extending the oligonucleotide extension primer in the extension solution by using the nucleic acid polymerizing enzyme to add a nucleotide analog to the oligonucleotide extension primer at the active site to form an extended oligonucleotide extension primer, wherein the nucleotide analog being added is complementary to the nucleotide of the target nucleic acid molecule at the active site;
determining the amounts of each type of the nucleotide analogs in the extension solution after said extending, each type being present in a second amount;
comparing the first and second amount of each type of the nucleotide analog; and
identifying the type of nucleotide analog where the first and second amounts differ as the nucleotide added to the oligonucleotide extension primer at the active site.

2. A method according to claim 1, wherein the target nucleic acid molecule is present in the sample as double stranded DNA.

3. A method according to claim 1, wherein the target nucleic acid molecule is present in the sample as single stranded DNA.

4. A method according to claim 1, wherein the target nucleic acid molecule is present in the sample as RNA.

5. A method according to claim 1, wherein said oligonucleotide amplification primer and said oligonucleotide extension primer have different melting temperatures (Tₘ).

6. A method according to claim 1, wherein the phosphatase is calf intestinal alkaline phosphatase, shrimp alkaline phosphatase, and mixtures thereof.

7. A method according to claim 1, wherein said subjecting the sample to a polymerase chain reaction process involves use of oligonucleotide amplification primers, said method further comprising:
digesting the oligonucleotide amplification primers which do not produce the amplification product during said subjecting the sample to a polymerase chain reaction process with exonuclease I and
inactivating the exonuclease I.

8. A method according to claim 1, wherein said subjecting the sample to a polymerase chain reaction process involves use of oligonucleotide amplification primers, one of which contains a 5′-phosphate group, said method further comprising:
subjecting the amplification product, prior to said subjecting the amplification product to treatment with a phosphatase, with λ-exonuclease under conditions effective to digest one strand of the amplification product containing the 5′-phosphate group; and
inactivating the λ-exonuclease.

9. A method according to claim 1, wherein each type of nucleotide analog is a dideoxy nucleotide analog.

10. A method according to claim 1, wherein said determining is carried out by electrospraying the extension solution.

11. A method according to claim 10, wherein said electrospraying is carried out with an electrospray device comprising:
a substrate having an injection surface and an ejection surface opposing the injection surface, wherein the substrate is an integral monolith comprising:
an entrance orifice on the injection surface;
an exit orifice on the ejection surface;
a channel extending between the entrance orifice and the exit orifice; and
a recess extending into the ejection surface and surrounding the exit orifice, thereby defining a nozzle on the ejection surface.

12. A method according to claim 11, wherein said substrate has a plurality of entrance orifices on the injection surface, a plurality of exit orifices on the ejection surface with each of the plurality of exit orifices corresponding to a respective one of the plurality entrance orifices, and a plurality of channels extending between one of the plurality of exit orifices and the corresponding one of the plurality of entrance orifices.

13. A method according to claim 11, wherein the electrospray device further comprises:
a voltage application system comprising:
a first electrode attached to said substrate to impart a first potential to said substrate and
a second electrode to impart a second potential, wherein the first and the second electrodes are positioned to define an electric field surrounding the exit orifice.

14. A method according to claim 13, wherein the first electrode is electrically insulated from fluid passing through said electrospray device and the second potential is applied to the fluid.

15. A method according to claim 13, wherein the first electrode is in electrical contact with fluid passing through said electrospray device and the second electrode is positioned on the ejection surface.

16. A method according to claim 13, wherein application of potentials to said first and second electrodes causes fluid passing through said electrospray device fluid to discharge from the exit orifice in the form of a spray.

17. A method according to claim 13, wherein application of potentials to said first and second electrodes causes fluid passing through said electrospray device fluid to discharge from the exit orifice in the form of droplets.

18. A method according to claim 11, wherein said electrospray device further comprises:
a porous polymeric material associated with said electrospray device at a location suitable to effect liquid chromatographic separation of materials passing through said electrospray device.

19. A method according to claim 10, wherein said determining further comprises detecting the amounts of each type of the nucleotide analogs in the electrospray.

20. A method according to claim 19, wherein said detecting is carried out by mass spectrometry, fluorescence, ion conductivity, liquid chromatography, capillary electrophoresis, radioactive assay, NMR, ELISA, and combinations thereof.

21. A method according to claim 10 further comprising:
passing the extension solution through a metal chelating resin prior to said electrospraying.

22. A method according to claim 1, wherein said method is carried out in a single container.

23. A method of detecting single nucleotide polymorphisms comprising:
providing a sample potentially containing a target nucleic acid molecule;
subjecting the sample to a polymerase chain reaction process involving use of oligonucleotide amplification primers under conditions effective to amplify any of the target nucleic acid molecule present in the sample to produce an amplification product;
passing the amplification product through a molecular weight filter configured to retain amplified target nucleic acid molecule but not the amplification primers;
providing an oligonucleotide extension primer complementary to a portion of the target nucleic acid molecule;
providing a nucleic acid polymerizing enzyme;
providing a plurality of types of nucleotide analogs;
blending the retained target nucleic acid molecule, the oligonucleotide extension primer, the nucleic acid polymerizing enzyme, and the nucleotide analogs, each type being present in a first amount, to form an extension solution where the oligonucleotide extension primer is hybridized to the target nucleic acid molecule to form a primed target nucleic acid molecule and the nucleic acid polymerizing enzyme is positioned to add nucleotide analogs to the primed target nucleic acid molecule at an active site;
extending the oligonucleotide extension primer in the extension solution by using the nucleic acid polymerizing enzyme to add a nucleotide analog to the oligonucleotide extension primer at the active site to form an extended oligonucleotide extension primer, wherein the nucleotide analog being added is complementary to the nucleotide of the target nucleic acid molecule at the active site;
determining the amounts of each type of the nucleotide analogs in the extension solution after said extending, each type being present in a second amount;
comparing the first and second amount of each type of the nucleotide analog; and
identifying the type of nucleotide analog where the first and second amounts differ as the nucleotide added to the oligonucleotide extension primer at the active site.

24. A method according to claim 23, wherein the target nucleic acid molecule is present in the sample as double stranded DNA.

25. A method according to claim 23, wherein the target nucleic acid molecule is present in the sample as single stranded DNA.

26. A method according to claim 23, wherein the target nucleic acid molecule is present in the sample as RNA.

27. A method according to claim 23, wherein said oligonucleotide amplification primer and said oligonucleotide extension primer have different melting temperatures (Tₘ).

28. A method according to claim 23, wherein said subjecting the sample to a polymerase chain reaction process involves use of oligonucleotide amplification primers, one of which contains a 5′-phosphate group, said method further comprising:
subjecting the amplification product, prior to said passing the amplification product through a molecular weight filter, with λ-exonuclease under conditions effective to digest one strand of the amplification product containing the 5′-phosphate group; and
inactivating the λ-exonuclease.

29. A method according to claim 23, wherein said passing the amplification product is vaccum-assisted.

30. A method according to claim 23, wherein each type of nucleotide analog is a dideoxy nucleotide analog.

31. A method according to claim 23 wherein said determining is carried out by electrospraying the extension solution.

32. A method according to claim 31, wherein said electrospraying is carried out with an electrospray device comprising:
a substrate having an injection surface and an ejection surface opposing the injection surface, wherein the substrate is an integral monolith comprising:
an entrance orifice on the injection surface;
an exit orifice on the ejection surface;
a channel extending between the entrance orifice and the exit orifice; and
a recess extending into the ejection surface and surrounding the exit orifice, thereby defining a nozzle on the ejection surface.

33. A method according to claim 32, wherein said substrate has a plurality of entrance orifices on the injection surface, a plurality of exit orifices on the ejection surface with each of the plurality of exit orifices corresponding to a respective one of the plurality entrance orifices, and a plurality of channels extending between one of the plurality of exit orifices and the corresponding one of the plurality of entrance orifices.

34. A method according to claim 32, wherein the electrospray device further comprises:
a voltage application system comprising:
a first electrode attached to said substrate to impart a first potential to said substrate; and
a second electrode to impart a second potential, wherein the first and the second electrodes are positioned to define an electric field surrounding the exit orifice.

35. A method according to claim 34, wherein the first electrode is electrically insulated from fluid passing through said electrospray device and the second potential is applied to the fluid.

36. A method according to claim 34, wherein the first electrode is in electrical contact with fluid passing through said electrospray device and the second electrode is positioned on the ejection surface.

37. A method according to claim 34, wherein application of potentials to said first and second electrodes causes fluid passing through said electrospray device fluid to discharge from the exit orifice in the form of a spray.

38. A method according to claim 34, wherein application of potentials to said first and second electrodes causes fluid passing through said electrospray device fluid to discharge from the exit orifice in the form of droplets.

39. A method according to claim 32, wherein said electrospray device further comprises:
a porous polymeric material associated with said electrospray device at a location suitable to effect liquid chromatographic separation of materials passing through said electrospray device.

40. A method according to claim 31, wherein said determining further comprises detecting the amounts of each type of the nucleotide analogs in the electrospray.

41. A method according to claim 40, wherein said detecting is carried out by mass spectrometry, fluorescence, ion conductivity, liquid chromatography, capillary electrophoresis, radioactive assay, NMR, ELISA, and combinations thereof.

42. A method according to claim 31 further comprising:
passing the extension solution through a metal chelating resin prior to said electrospraying.

43. A method according to claim 23, wherein said method is carried out in a single container.

44. A method of detecting single nucleotide polymorphisms comprising:
providing a sample potentially containing a target nucleic acid molecule;
subjecting the sample to a polymerase chain reaction process involving use of oligonucleotide amplification primers under conditions effective to amplify any of the target nucleic acid molecule present in the sample to produce an amplification product;
subjecting the amplification product to treatment with a phosphatase under conditions effective to remove 5′ phosphates from free dNTPs in the amplification product;
inactivating the phosphatase;
providing an oligonucleotide extension primer complementary to a portion of the target nucleic acid molecule;
providing a nucleic acid polymerizing enzyme;
providing a plurality of types of nucleotide analogs;
blending the amplification product treated with a phosphatase, the oligonucleotide extension primer, the nucleic acid polymerizing enzyme, and the nucleotide analogs to form an extension solution where the oligonucleotide extension primer is hybridized to the target nucleic acid molecule to form a primed target nucleic acid molecule and the nucleic acid polymerizing enzyme is positioned to add nucleotide analogs to the primed target nucleic acid molecule at an active site;
extending the oligonucleotide extension primer in the extension solution by using the nucleic acid polymerizing enzyme to add a nucleotide analog to the oligonucleotide extension primer at the active site to form an extended oligonucleotide extension primer, wherein the nucleotide analog being added is complementary to the nucleotide of the target nucleic acid molecule at the active site; and
determining the nucleotide analog added to the oligonucleotide extension primer at the active site by electrospraying the extended oligonucleotide extension primer.

45. A method according to claim 44, wherein the target nucleic acid molecule is present in the sample as double stranded DNA.

46. A method according to claim 44, wherein the target nucleic acid molecule is present in the sample as single stranded DNA.

47. A method according to claim 44, wherein the target nucleic acid molecule is present in the sample as RNA.

48. A method according to claim 44, wherein said oligonucleotide amplification primer and said oligonucleotide extension primer have different melting temperatures (Tₘ).

49. A method according to claim 44, wherein the phosphatase is calf intestinal alkaline phosphatase, shrimp alkaline phosphatase, and mixtures thereof.

50. A method according to claim 44, wherein said subjecting the sample to a polymerase chain reaction process involves use of oligonucleotide amplification primers, said method further comprising:
digesting the oligonucleotide amplification primers which do not produce the amplification product during said subjecting the sample to a polymerase chain reaction process with exonuclease I and
inactivating the exonuclease I.

51. A method according to claim 44, wherein said subjecting the sample to a polymerase chain reaction process involves use of oligonucleotide amplification primers, one of which contains a 5′-phosphate group, said method further comprising:
subjecting the amplification product with λ-exonuclease, prior to said subjecting the amplification product to treatment with a phosphatase, under conditions effective to digest one strand of the amplification product containing the 5′-phosphate group; and
inactivating the λ-exonuclease.

52. A method according to claim 44, wherein each type of nucleotide analog is a dideoxy nucleotide analog.

53. A method according to claim 44, wherein said electrospraying is carried out with an electrospray device comprising:
a substrate having an injection surface and an ejection surface opposing the injection surface, wherein the substrate is an integral monolith comprising:
an entrance orifice on the injection surface;
an exit orifice on the ejection surface;
a channel extending between the entrance orifice and the exit orifice; and
a recess extending into the ejection surface and surrounding the exit orifice, thereby defining a nozzle on the ejection surface.

54. A method according to claim 53, wherein said substrate has a plurality of entrance orifices on the injection surface, a plurality of exit orifices on the ejection surface with each of the plurality of exit orifices corresponding to a respective one of the plurality entrance orifices, and a plurality of channels extending between one of the plurality of exit orifices and the corresponding one of the plurality of entrance orifices.

55. A method according to claim 53, wherein the electrospray device further comprises:
a voltage application system comprising:
a first electrode attached to said substrate to impart a first potential to said substrate and
a second electrode to impart a second potential, wherein the first and the second electrodes are positioned to define an electric field surrounding the exit orifice.

56. A method according to claim 55, wherein the first electrode is electrically insulated from fluid passing through said electrospray device and the second potential is applied to the fluid.

57. A method according to claim 55, wherein the first electrode is in electrical contact with fluid passing through said electrospray device and the second electrode is positioned on the ejection surface.

58. A method according to claim 55, wherein application of potentials to said first and second electrodes causes fluid passing through said electrospray device fluid to discharge from the exit orifice in the form of a spray.

59. A method according to claim 55, wherein application of potentials to said first and second electrodes causes fluid passing through said electrospray device fluid to discharge from the exit orifice in the form of droplets.

60. A method according to claim 53, wherein said electrospray device further comprises:
a porous polymeric material associated with said electrospray device at a location suitable to effect liquid chromatographic separation of materials passing through said electrospray device.

61. A method according to claim 44, wherein said determining further comprises detecting the amounts of each type of the nucleotide analogs in the electrospray.

62. A method according to claim 61, wherein said detecting is carried out by mass spectrometry, fluorescence, ion conductivity, liquid chromatography, capillary electrophoresis, MALDI-TOF, radioactive assay, NMR, ELISA, and combinations thereof.

63. A method according to claim 44 further comprising:
passing the extension solution through a molecular weight filter configured to separate the amplified target nucleic acid molecule and the extended oligonucleotide extension primers from low molecular weight reaction components, prior to said electrospraying.

64. A method according to claim 63, wherein said passing the extension solution is vacuum-assisted.

65. A method according to claim 44, wherein said method is carried out in a single container.

66. A method of detecting single nucleotide polymorphisms comprising:
providing a sample potentially containing a target nucleic acid molecule;
subjecting the sample to a polymerase chain reaction process involving use of oligonucleotide amplification primers under conditions effective to amplify any of the target nucleic acid molecule present in the sample to produced an amplification product;
passing the amplification product through a molecular weight filter configured to retain amplified target nucleic acid molecule but not the amplification primers;
providing an oligonucleotide extension primer complementary to a portion of the target nucleic acid molecule;
providing a nucleic acid polymerizing enzyme;
providing a plurality of types of nucleotide analogs;
blending the retained target nucleic acid molecule, the oligonucleotide extension primer, the nucleic acid polymerizing enzyme, and the nucleotide analogs to form an extension solution where the oligonucleotide extension primer is hybridized to the target nucleic acid molecule to form a primed target nucleic acid molecule and the nucleic acid polymerizing enzyme is positioned to add nucleotide analogs to the primed target nucleic acid molecule at an active site;
extending the oligonucleotide extension primer in the extension solution by using the nucleic acid polymerizing enzyme to add a nucleotide analog to the oligonucleotide extension primer at the active site to form an extended oligonucleotide extension primer, wherein the nucleotide analog being added is complementary to the nucleotide of the target nucleic acid molecule at the active site; and
determining the nucleotide analog added to the oligonucleotide extension primer at the active site by electrospraying the extended oligonucleotide extension primer.

67. A method according to claim 66, wherein the target nucleic acid molecule is present in the sample as double stranded DNA.

68. A method according to claim 66, wherein the target nucleic acid molecule is present in the sample as single stranded DNA.

69. A method according to claim 66, wherein the target nucleic acid molecule is present in the sample as RNA.

70. A method according to claim 66, wherein said oligonucleotide amplification primer and said oligonucleotide extension primer have different melting temperatures (Tₘ).

71. A method according to claim 66, wherein said subjecting the sample to a polymerase chain reaction process involves use of oligonucleotide amplification primers, one of which contains a 5′-phosphate group, said method further comprising:
subjecting the amplification product, prior to said passing the amplification product through a molecular weight filter, with λ-exonuclease under conditions effective to digest one strand of the amplification product containing the 5′-phosphate group; and
inactivating the λ-exonuclease.

72. A method according to claim 66, wherein said passing the amplification product is vacuum-assisted.

73. A method according to claim 66, wherein each type of nucleotide analog is a dideoxy nucleotide analog.

74. A method according to claim 66, wherein said electrospraying is carried out with an electrospray device comprising:
a substrate having an injection surface and an ejection surface opposing the injection surface, wherein the substrate is an integral monolith comprising:
an entrance orifice on the injection surface;
an exit orifice on the ejection surface;
a channel extending between the entrance orifice and the exit orifice; and
a recess extending into the ejection surface and surrounding the exit orifice, thereby defining a nozzle on the ejection surface.

75. A method according to claim 74, wherein said substrate has a plurality of entrance orifices on the injection surface, a plurality of exit orifices on the ejection surface with each of the plurality of exit orifices corresponding to a respective one of the plurality entrance orifices, and a plurality of channels extending between one of the plurality of exit orifices and the corresponding one of the plurality of entrance orifices.

76. A method according to claim 74, wherein the electrospray device further comprises:
a voltage application system comprising:
a first electrode attached to said substrate to impart a first potential to said substrate and
a second electrode to impart a second potential, wherein the first and the second electrodes are positioned to define an electric field surrounding the exit orifice.

77. A method according to claim 76, wherein the first electrode is electrically insulated from fluid passing through said electrospray device and the second potential is applied to the fluid.

78. A method according to claim 76, wherein the first electrode is in electrical contact with fluid passing through said electrospray device and the second electrode is positioned on the ejection surface.

79. A method according to claim 76, wherein application of potentials to said first and second electrodes causes fluid passing through said electrospray device fluid to discharge from the exit orifice in the form of a spray.

80. A method according to claim 76, wherein application of potentials to said first and second electrodes causes fluid passing through said electrospray device fluid to discharge from the exit orifice in the form of droplets.

81. A method according to claim 74, wherein said electrospray device further comprises:
a porous polymeric material associated with said electrospray device at a location suitable to effect liquid chromatographic separation of materials passing through said electrospray device.

82. A method according to claim 66, wherein said determining further comprises detecting the amounts of each type of the nucleotide analogs in the electrospray.

83. A method according to claim 82, wherein said detecting is carried out by mass spectrometry, fluorescence, ion conductivity, liquid chromatography, capillary electrophoresis, MALDI-TOF, radioactive assay, NMR, ELISA, and combinations thereof.

84. A method according to claim 66 further comprising:
passing the extension solution through a metal chelating resin prior to said electrospraying.

85. A method according to claim 66 further comprising:
passing the extension solution through a molecular weight filter configured to separate the amplified target nucleic acid molecule and the extended oligonucleotide extension primers from low molecular weight reaction components, prior to said electrospraying.

86. A method according to claim 85, wherein said passing the extension solution is vacuum-assisted.

87. A method according to claim 66, wherein said method is carried out in a single container.
